(19) United States
(12) Patent Application Publication
(10) Pub. No.: US 2011/0212986 A1
(43) Pub. Date: Sep. 1, 2011

(54) ANTI-FLAVIVIRUS THERAPEUTIC

(75) Inventors: Pei-Yong Shi, Glenmont, NY (US); Francesc Puig-Basagoiti, Allston, MA (US); Krzysztof W. Pankiewicz, Minneapolis, MN (US); Krzysztof Felczak, Minneapolis, MN (US); Liqiang Chen, Shoreview, MN (US)

(73) Assignees: HEALTH RESEARCH, INC., Menands, NY (US); REGENTS OF THE UNIVERSITY OF MINNESOTA, St. Paul, MN (US)

(21) Appl. No.: 13/060,035

(22) PCT Filed: Aug. 20, 2009
(86) PCT No.: PCT/US09/54468

§ 371 Date: May 10, 2011

Related U.S. Application Data
(60) Provisional application No. 61/090,639, filed on Aug. 21, 2008.

(51) Int. Cl. A61K 31/475; C07D 491/16; A61P 31/14; C12N 7/00

(52) U.S. Cl. 514/283; 546/48; 435/235.1

(57) ABSTRACT

The present invention relates to anti-flavivirus compounds, including lycorine and derivatives thereof, and their use in treating a subject infected by a flavivirus. The present invention also relates to the use of the anti-flavivirus compounds for the prophylaxis of flavivirus infection. The present invention further relates to a method of suppressing viral RNA synthesis of a flavivirus. Also described is a method of preparing an anti-flavivirus compound for use in the treatment or prophylaxis of flavivirus infection.

The present application claims benefit of U.S. Provisional Patent Application Ser. No. 61/090,639, filed Aug. 21, 2008, which is hereby incorporated by reference in its entirety.

### GOVERNMENT RIGHTS STATEMENT

The present invention was made with U.S. Government support under National Institute of Allergy and Infectious Disease/National Institutes of Health Grant No. NOI-AI-25490 and National Institutes of Health Grant Nos. U01-AI061193 and U54-AI057158. The U.S. Government has certain rights in the invention.

### FIELD OF THE INVENTION

The present invention relates to anti-flavivirus compounds, methods of making these compounds, the use of these compounds for the treatment or prophylaxis of flavivirus infection and for the suppression or inhibition of flavivirus activity.

### BACKGROUND OF THE INVENTION

The family Flaviviridae consists of three genera, the flaviviruses, the pestiviruses, and the hepatitis C viruses. Many members of the genus Flavivirus are arthropod-borne human pathogens, including four serotypes of dengue virus (DENV), West Nile virus (WNV), Japanese encephalitis virus (JEV), yellow fever virus (YFV), and tick-borne encephalitis virus (TBEV) (Gubleret al., 2007). More than 50 million, 200,000, and 50,000 human cases were reported annually for DENV, YFV, and JEV infections, respectively (Gubler et al., 2007). Since the initial outbreak of WNV in New York in 1999, the virus has caused thousands of human cases of infections each year and has spread throughout North America (Kramer et al., 2007).

No effective antiviral therapy has been approved for the treatment of flavivirus infections. Human vaccines are currently available only for JEV, YFV, and TBEV. It has been well recognized that development of a vaccine for DENV is particularly challenging, because of the need to simultaneously immunize against all four DENV serotypes.

Therefore, development of therapeutics is the priority for intervention in flavivirus infections. The present invention is directed to overcome these and other deficiencies in the art.

### SUMMARY OF THE INVENTION

In one aspect, the invention relates to anti-flavivirus compounds having the structure (I), (II), or (III), as follows:

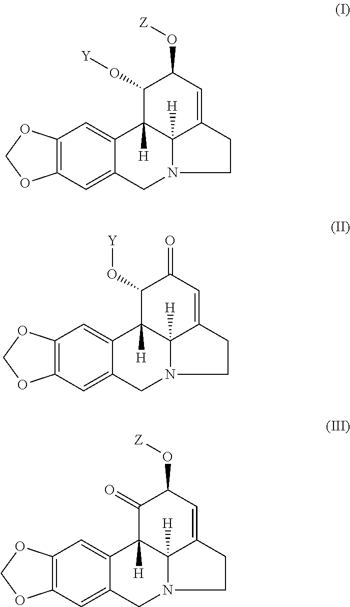

wherein Y and Z are each independently selected from the group consisting of H, alkyl, aralkyl, alkoxyalkyl, heteroalkyl, alkenyl, acyl, alkylsilyl, and arylalkylsilyl; or Y and Z together are alkylidenyl or aralkylidenyl.

In another aspect, the invention relates to the use of the anti-flavivirus compounds of the present invention in a method of treating a subject infected by a flavivirus. This method involves administering to a subject infected by a flavivirus an effective amount of a compound of the structure (I), (II), or (III), or a pharmaceutically acceptable salt of the compound, optionally in combination with a pharmaceutically acceptable excipient, carrier, or additive.

In another aspect, the invention relates to the use of the anti-flavivirus compounds of the present invention in a method of preventing a flavivirus infection in a subject. This method involves administering to a subject an effective amount of a compound of the structure (I), (II), or (III), or a pharmaceutically acceptable salt of the compound, optionally in combination with a pharmaceutically acceptable excipient, carrier, or additive.

In a further aspect, the invention relates to a method of suppressing viral RNA synthesis of a flavivirus. This method involves providing an anti-flavivirus compound of the present invention, and contacting the flavivirus with an effective amount of the compound to suppress the viral RNA synthesis of the flavivirus.

In yet another aspect, the invention relates to a method for preparing an anti-flavivirus compound for use in the treatment or prophylaxis of a flavivirus infection in a subject. This method involves providing a lycorine compound having a structure of:

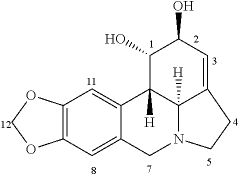

or a precursor of this compound. The method also involves substituting the hydroxyl group at position 1 and/or at position 2 of the lycorine compound, or precursor thereof, with a protecting group in order to yield an anti-flavivirus agent having a therapeutic index (TI) of 10 or greater, where the therapeutic index refers to the ratio of CC₅₀ (μM)/EC₅₀ (μM).

In another aspect, the invention relates to a novel anti-flavivirus compound having a structure of:

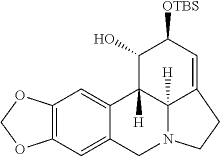

These, and other objects, features, and advantages of this invention will become apparent from the following detailed description of the various aspects of the invention taken in conjunction with the accompanying drawings.

### BRIEF DESCRIPTION OF THE DRAWINGS

FIGS. 1A-1C illustrate the identification of lycorine as an inhibitor of WNV and DENV-1 VLP infections. FIG. 1A shows the structure of lycorine. The carbon positions of the lycorine molecule are numbered. FIG. 1B is a schematic showing the production of flavivirus VLPs. Flavivirus VLPs were prepared by sequential transfection of BHK-21 cells with a luciferase-reporting replicon (Flavi-Rluc2A-Rep) and an SFV vector expressing flavivirus structural proteins (SFV-Flavi-CprME). See Example 2 (Materials and Methods) for details. FIG. 1C is a graph showing the inhibition of WNV and DENv-1 VLP infections by lycorine. Vero cells were infected with WNV (1 FFU/cell) or DENV-1 (0.01 FFU/cell) VLPs in the presence of 1.5 μM lycorine. Luciferase activities were measured at 24 h post-infection. Average results of three independent experiments are shown.

FIGS. 2A-2B are graphs illustrating the cytotoxicity and potency of lycorine against an epidemic strain of WNV. FIG. 2A: Cytotoxicity of lycorine in Vero cells. Cytotoxicity was examined by incubation of Vero cells with the indicated concentrations of lycorine. Cell viability was measured by an MTT assay, and is presented as a percentage of colorimetric absorbance derived from the compound-treated cells compared with that from the mock-treated cells (with 1% DMSO). Average results from three experiments are shown. FIG. 2B: Inhibition of WNV infection in cell culture. Vero cells were infected with an epidemic strain of WNV (0.1 MOI). The infected cells were immediately treated with lycorine at the indicated concentrations. Viral titers in culture fluids at 42 h p.i. were determined by plaque assays.

FIGS. 3A-3D are graphs illustrating the antiviral spectrum of lycorine. Viral titer reduction assays were performed for DENV-2 (FIG. 3A), YFV (FIG. 3B), WEEV (FIG. 3C), and VSV (FIG. 3D) in the presence of various concentrations of lycorine. (See details in Example 2, Materials and Methods.)

FIGS. 4A-4C are graphs illustrating the mechanism of lycorine-mediated inhibition of flaviviruses. FIG. 4A: Antiviral activities in Vero cells containing Rluc-Neo-Rep of WNV (left) and DENV-1 (right). Vero cells containing a WNV or DENV-1 replicon (Rluc-Neo-Rep) were treated with lycorine at the indicated concentrations, and were measured for luciferase activities at 24 or 48 h post-treatment. FIG. 4B: Analysis of lycorine using transient replicons of WNV (left) and DENV-1 (right). Luciferase replicons (Rluc2A-Rep) of WNV or DENV-1 were electroporated into BHK-21 cells. The transfected cells were immediately incubated with 1.5 μM lycorine, and were measured for luciferase activities at the indicated time points post-transfection. Numbers above the lycorine-treated datum points indicate percentages of luciferase signals from the compound-treated transfection compared with those from the mock-treated transfection (set to 100%). Error bars indicate the standard deviations from three independent experiments. FIG. 4C: Time-of-addition analysis of lycorine in WNV infection. Vero cells were infected with WNV at an MOI of 10 at 4° C. for 1 h. The infected cells were washed three times with PBS. Lycorine (1.2 μM) was then added to the cells at the indicated time points post-infection. The supernatants were assayed for viral titers at 20 h post-infection. As controls. 1% DMSO was added to the infected cells at 0, 12 and 20 h p.i. for estimation of its effect on viral production.

FIGS. 5A-5C illustrate the selection and characterization of lycorine-resistant WNV. FIG. 5A: Scheme for selection of lycorine-resistant WNV. Three independent selections were performed. P1 through P6 were selected at 0.8 μM lycorine; P7 through P12 were selected at 1.2 μM. FIG. 5B: Resistance profile during selection. Viruses from each of the 12 passages were monitored for their resistance. Vero cells were infected with viruses at an MOI of 0.1 in the presence of 0.8 μM lycorine (P1-P6), 1.2 μM lycorine (P7-P12), or 1% DMSO (as a negative control). At 42 h p.i., viral titers in culture fluids were quantified by plaque assays. Resistance is quantified by comparison of the viral titers from the lycorine-treated infections with the viral titers from the mock-treated infections. Results for representative passages from the three selections are shown. FIG. 5C: Plaque morphologies of WT and lycorine-resistant viruses. Plaque assays for WT and P12 viruses were performed on Vero cells in the absence of lycorine.

FIGS. 6A-6D illustrate the identification of a single-amino acid change in the 2K peptide as a resistance determinant. FIG. 6A: Summary of mutations identified from the three selections. Locations of the nucleotide and/or amino acid channels are indicated. Mixed populations (containing both the WT-nucleotide and the indicated mutant-nucleotide) were found in the E gene from selections I and II. FIG. 6B: Plaque morphologies of WT and recombinant C1161U, U1789C, A1287C, C1418U, and G6871A viruses. Plaques were developed in the absence of lycorine. FIG. 6C: Resistance analyses of WT virus, three independently selected P12 viruses (from Sel, I, II, and III), and recombinant C1161U, U1789C, A1287C, C1418U, and G6871A viruses. The resistance assays were performed as described in for FIG. 5B. FIG. 6D: Alignment of amino acid sequences of flavivirus 2K peptide. The 23-amino acid sequences of 2K peptide are aligned for nine flaviviruses. The conserved Val residues in viruses from JEV- and DENV-serocomplexes are shaded in grey. The 2K peptide sequences of WNV, KUNV, JEV, DENV-1, DENV-2, DENV-3, DENV-4, YFV, and TBEV are derived from GenBank accession numbers AF404756, D00246, AF315119, U88535, M29095, M93130, AY947539, X03700, and AF069066, respectively. The 2K peptide amino acid sequences listed in FIG. 6D for the nine flaviviruses have been assigned sequence identifier numbers (i.e., SEQ ID NOs), as follows: SEQ ID NO:1 (SQTDNQLAVFLICVMTLVSAVAA) (=WNV 2K peptide); SEQ ID NO:2 (SQTDNQLAVFLICVLTLVGAVAA) (=KUNV 2K peptide); SEQ ID NO:3 (SQTDNQLAVFLICVLTVVGVVAA) (=JEV 2K peptide); SEQ ID NO:4 (TPQDNQLAYVVIGLLFMILTAAA) (=DENV-1 2K peptide); SEQ ID NO:5 (TPQDNQLTYVVIAILTVVAATMA) (=DENV-2 2K peptide); SEQ ID NO:6 (TPQDNQLAYVVIGILTLAAIVAA) (=DENV-3 2K peptide); SEQ ID NO:7 (TPQDNQLIYVILTILTIIGLIAA) (=DENV-4 2K peptide); SEQ ID NO:8 (SIQDNQVAYLIIGILTLVSAVAA) (=YFV 2K peptide); and SEQ ID NO:9 (SSDDNKLAYFLLTLCSLAGLVAA) (=TBEV 2K peptide).

FIGS. 7A-7B illustrate the replication kinetics of WT and 2K-mutant viruses in the presence and absence of lycorine. Vero cells, in a 12-well plate, were infected with WNV (WT and G6871A MT) at an MOI of 5, incubated at 4° C. for 1 h, washed three times with PBS, and incubated at 37° C. with medium containing 1.2-1 μM lycorine or with medium containing 1% DMSO. At 16, 24, 36, and 48 h p.i., viral titers in supernatants were determined by plaque assays (FIG. 7A). The infected cells were washed twice with PBS, lysed with 250 μl of lysis buffer, and frozen at 80° C. The cell lysates (10 μl) were analyzed by western blotting (FIG. 7B). Four monoclonal antibodies against NS1 (1:1000 dilution; purchased from Sigma), NS3 (1:4000 dilution; in-house generated), NS5 (1:4000 dilution; in-house generated), or β-actin (1:1000 dilution; purchased from Chemicon) were mixed and used as primary antibodies. Horse radish peroxidase (HRP)-labeled anti-mouse IgG (1:4000 dilution) was used as a secondary antibody. β-actin was used as a loading control. One representative of two experiments is shown.

FIGS. 8A-8B are graphs illustrating the enhancement of viral replication through mutation of the 2K peptide. FIG. 8A: Resistance and replication analyses using WNV replicon. The effect of the G6871A mutation in the 2K peptide on resistance to lycorine was quantified using a transient replicon (Rluc2A-Rep) assay. Equal amounts (10 μg) of WT and G6871A mutant replicon RNAs were electroporated into BHK-21 cells. The transfected cells were immediately treated with lycorine (1.5 μM) or without lycorine (1% DMSO as controls). Luciferase activities were measured at the indicated time points post-transfection. Average results from three independent experiments are presented. FIG. 8B: Growth kinetics of WT and 2K peptide G6871A mutant viruses. Vero cells (results shown in top graph) and C6/36 cells (results shown in bottom graph) were infected with the WT and the 2K peptide mutant viruses at an MOI of 0.05. After 1-h incubation, the cells were washed three times with PBS and the medium was replenished. Viral titers in culture fluids were quantified at the indicated time points using plaque assays.

### DETAILED DESCRIPTION OF THE INVENTION

Terms are used within their accepted meanings. The definitions provided hereinbelow are meant to clarify, but not limit, the terms defined. Throughout this specification, the terms and substituents retain their definitions.

The present invention relates to a class of compounds having antiviral activity against flaviviruses. This class of compounds is also referred to herein as “anti-flavivirus compounds” or the like.

As used herein, the term “flavivirus” includes all viruses in the Flavivirus genus. Specific examples of flaviviruses contemplated by the present invention include, but are not limited to, West Nile virus (WNV), dengue virus (DENV), Japanese encephalitis virus (JEV), yellow fever virus (YFV), tick-borne encephalitis virus (TBEV), St. Louis encephalitis virus (SLEV), Alfuy virus (AV), Koutango virus (KV), Kunjin virus (KUNV), Cacipacore virus (CV), Yaounde virus (YV), and Murray Valley encephalitis virus (MVEV).

The flavivirus genome is a plus-sense, single-stranded RNA of about 11,000 nucleotides (Lindenbach et al., 2007). The genomic RNA consists of a 5′ untranslated region (UTR), a single open reading frame (ORF), and a 3′UTR. The single ORF encodes a long polyprotein that is co-translationally and post-translationally processed by viral and host proteases into ten mature viral proteins. The N-terminus of the polyprotein contains three structural proteins: capsid (C), premembrane (prM/M), and envelope (E). The C-terminus of the polyprotein contains seven nonstructural (NS) proteins: NS1, NS2A, NS2B, NS3, NS4A, NS4B, and NS5. Complete cleavage of the polyprotein generates a 2K peptide between NS4A and NS4B. The 2K peptide spans the membrane of endoplasmic reticulum (ER). Two viral proteins have enzymatic activities. NS3 functions as a protease (with NS2B as a cofactor), a nucleotide triphosphatase, an RNA triphosphatase, and a helicase (Falgout, Miller, and Lal, 1993; Li et al., 1999; Warrener et al., 1993; Wengler and Wengler, 1991). NS5 acts as a methyltransferase (MTase) and an RNA-dependent-RNA polymerase (RdRp) (Ackermann and Padmanabhan. 2001; Egloff et al., 2002; Guyatt et al., 2001; Rayet al., 2006; Tan et al., 1996). Besides functioning in replication, flaviviral NS proteins play roles in virion assembly (Jones et al., 2005; Kummerer and Rice, 2002: Leung et al., 2001; Liu et al., 2003) and evasion of host innate immune responses (Best et al., 2005: Guo et al., 2005; Liu et al., 2005: Munoz-Jordan et al., 2005: Munoz-Jordan et al., 2003). Upon viral entry and nucleocapsid uncoating, the genomic RNA is translated into proteins, which are translocated across the ER membrane to form the replication complexes (Lindenbach et al., 2007). The molecular details of individual NS proteins and their roles in flavivirus replication remain to be characterized.

The class of anti-flavivirus compounds of the present invention includes lycorine and derivatives thereof, as further described herein. As used herein, the terms “lycorine compound,” “lycorine compound derivative,” and the like are meant to be used interchangebly with the term “anti-flavivirus compound.”

Lycorine is an alkaloid compound found in several plants, such as daffodil (Narcissus pseudonarcissus) and bush lily (Clivia miniata). A number of biological activities have been reported for lycorine, including inhibition of protein and DNA synthesis (Chattopadhyay et al., 1984), cell growth and division (De Leo et al., 1973), and anti-leukemia effect (Liu et al., 2004, 2007). In addition, the compound has been shown to inhibit poliovirus (Ieven et al., 1982), Severe Acute Respiratory Syndrome-associated coronavirus (SARS-CoV) (Li et al., 2005), herpes simplex virus (type 1) (Renard-Nozaki et al., 1989), and vaccinia virus (Zhou et al., 2003). However, until the present invention, lycorine, or deriviatives thereof, has not been described or shown to have any antiviral activity against flaviviruses.

In accordance with the present invention, the inventors have determined that lycorine inhibits flaviviruses with a selective antiviral spectrum. Mode-of-action analysis indicates that lycorine inhibits flaviviruses mainly through suppression of viral RNA synthesis. Structural modifications of the lycorine compound increased its potency while decreasing its cytotoxicity, indicating the compound's efficacy as a therapeutic for use against flavivirus infection in mammals, including, for example, humans. Furthermore, the inventors determined that a single-amino acid substitution in WNV 2K peptide confers resistance to lycorine, partially through enhancement of viral RNA synthesis, thus revealing a direct role of the 2K peptide in flavivirus RNA replication.

In one embodiment, the anti-flavivirus compounds of the present invention are compounds having the structure (I), (II), or (III), as follows:

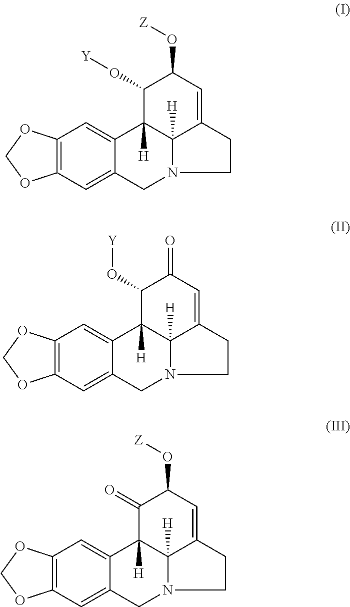

wherein Y and Z are each independently selected from the group consisting of H, alkyl, aralkyl, alkoxyalkyl, heteroalkyl, alkenyl, acyl, alkylsilyl, and arylalkylsilyl; or Y and Z together are alkylidenyl or aralkylidenyl.

In one embodiment, the anti-flavivirus compounds of the present invention have the structures (I), (II), or (III), wherein Y and Z are each independently selected from the group consisting of: H, methoxymethyl (MOM), tetrahydropyranyl (THP), allyl (All), benzyl (Bzyl), t-butyl (tBu), t-butyldimethylsilyl (TBS), t-butyldiphenylsilyl (TBDPS), acetyl (Ac), pivalyl (Piv), and benzoyl (Bzoyl); or wherein Y and Z together are isopropylidenyl [(CH₃)₂CH] or benzylidenyl [Φ-CH].

In another embodiment, the anti-flavivirus compounds of the present invention have the structures (I), (II), or (III), wherein at least one of Y or Z is selected from the group consisting of: methyl, ethyl, methoxymethyl (MOM), tetrahydropyranyl (THP), allyl (All), benzyl (Bzyl), t-butyl (tBu), t-butyldimethylsilyl (TBS), t-butyldiphenylsilyl (TBDPS), acetyl (Ac), pivalyl (Piv), and benzoyl (Bzoyl).

In another embodiment, the anti-flavivirus compounds of the present invention have the structures (I), (II), or (III), wherein Y or Z is substituted acetyl or substituted benzoyl.

In a further embodiment, the anti-flavivirus compounds of the present invention have the structure (I), wherein Y and Z are each Ac.

In yet another embodiment, the anti-flavivirus compounds of the present invention have the structure (I), wherein Y is Ac and Z is H.

In another embodiment, the anti-flavivirus compounds of the present invention have the structure (I), wherein Y is H and Z is TBS.

In another embodiment, the anti-flavivirus compounds of the present invention have the structure (II), wherein Y is selected from the group consisting of: H, alkyl, aralkyl, alkoxyalkyl, heteroalkyl, alkenyl, acyl, alkylsilyl, and arylalkylsilyl.

In a further embodiment, the anti-flavivirus compounds of the present invention have the structure (II), wherein Y is selected from the group consisting of: methyl, ethyl, methoxymethyl (MOM), tetrahydropyranyl (THP), allyl (All), benzyl (Bzyl), t-butyl (tBu), t-butyldimethylsilyl (TBS), t-butyldiphenylsilyl (TBDPS), acetyl (Ac), pivalyl (Piv), and benzoyl (Bzoyl).

In another embodiment, the anti-flavivirus compounds of the present invention have the structure (II), wherein Y is substituted acetyl or substituted benzoyl.

In yet another embodiment, the anti-flavivirus compounds of the present invention have the structure (II), wherein Y is Ac.

The anti-flavivirus compounds of the present invention may be made by a variety of methods, including standard synthetic methods well known by those of ordinary skill in the chemical synthesis art. Illustrative general synthetic methods for the anti-flavivirus compounds of the present invention are set forth herein, including working Examples for particular compounds.

The present invention also relates to a pharmaceutically acceptable salt of the anti-flavivirus compounds described herein, optionally in combination with a pharmaceutically acceptable excipient, carrier, or additive. Suitable excipients, carriers, and additives for use with the anti-flavivirus compounds of the present invention are described herein, and others are well known in the art.

The present invention also relates to a method of treating a subject infected by a flavivirus. This method involves administering to a subject infected by a flavivirus an effective amount of a compound of the structure (I), (II), or (III), or a pharmaceutically acceptable salt of the compound, optionally in combination with a pharmaceutically acceptable excipient, carrier, or additive.

In another aspect, the invention relates to the use of the anti-flavivirus compounds of the present invention in a method of preventing a flavivirus infection in a subject. This method involves administering to a subject an effective amount of a compound of the structure (I), (II), or (III), or a pharmaceutically acceptable salt of the compound, optionally in combination with a pharmaceutically acceptable excipient, carrier, or additive.

The present invention further relates to a method of suppressing viral RNA synthesis of a flavivirus. This method involves providing an anti-flavivirus compound of the present invention, and contacting the flavivirus with an effective amount of the compound to suppress the viral RNA synthesis of the flavivirus.

The present invention also relates to a method for preparing an anti-flavivirus compound for use in the treatment or prophylaxis of a flavivirus infection in a subject. This method involves providing a lycorine compound having a structure of:

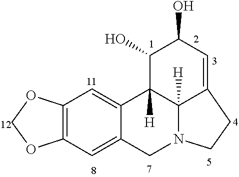

or a precursor of this compound. The method also involves substituting the hydroxyl group at position 1 and/or at position 2 of the lycorine compound, or precursor thereof, with a protecting group in order to yield an anti-flavivirus agent having a therapeutic index (TI) of 10 or greater, where the therapeutic index refers to the ratio of CC₅₀ (μM)/EC₅₀ (μM). The terms “CC₅₀” and “EC₅₀” are commonly known in the art to denote cytotoxicity and antiviral activity/potency, respectively.

In one embodiment of this method for preparing the anti-flavivirus compound, the protecting group is selected from the group consisting of: methoxymethyl (MOM), tetrahydropyranyl (THP), allyl (All), benzyl (Bzyl), t-butyl (tBu), t-butyldimethylsilyl (TBS), t-butyldiphenylsilyl (TBDPS), acetyl (Ac), pivalyl (Piv), benzoyl (Bzoyl), substituted acetyl, and substituted benzoyl.

The present invention also relates to a novel anti-flavivirus compound having a structure of:

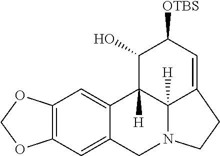

This novel anti-flavivirus compound is referred to herein as compound “1198.” An illustrative method of synthesizing this anti-flavivirus compound is found in Example 5 as set forth herein.

In one embodiment of anti-flavivirus compound 1198, the —OH group is protected by a protecting group selected from the group consisting of: methoxymethyl (MOM), tetrahydropyranyl (THP), allyl (All), benzyl (Bzyl), t-butyl (tBu), t-butyldimethylsilyl (TBS), t-butyldiphenylsilyl (TBDPS), acetyl (Ac), pivalyl (Piv), benzoyl (Bzoyl), substituted acetyl, and substituted benzoyl.

As used herein, the term “subject” is meant to refer to a mammal, and more particularly to a human.

An “alkyl” group refers to a saturated aliphatic hydrocarbon, including straight-chain, branched chain, and cyclic alkyl groups. Alkyl groups can comprise any combination of acyclic and cyclic subunits. Further, the term “alkyl” as used herein expressly includes saturated groups as well as unsaturated groups. Unsaturated groups contain one or more (e.g., one, two, or three) double bonds and/or triple bonds. The term “alkyl” includes substituted and unsubstituted alkyl groups. When substituted, the substituted group(s) may be hydroxyl, cyano, alkoxy, ═O, ═S, NO₂, N(CH₃)₂, amino, or SH. Preferably, the alkyl group has 1 to 12 carbons. More preferably, it is a lower alkyl of from 1 to 7 carbons. “Lower alkyl” refers to an alkyl group of one to six carbon atoms, i.e., C₁-C₆ alkyl. Examples of alkyl groups include, but are not limited to, methyl, ethyl, propyl, isopropyl, isobutyl, sec-butyl, tert-butyl, pentyl, n-hexyl, octyl, dodecyl, and the like.

An “alkenyl” group refers to an unsaturated hydrocarbon group containing at least one carbon-carbon double bond, including straight-chain, branched-chain, and cyclic groups. Preferably, the alkenyl group has 1 to 12 carbons. More preferably it is a lower alkenyl of from 1 to 7 carbons. The alkenyl group may be substituted or unsubstituted. When substituted, the substituted group(s) is preferably hydroxyl, cyano, alkoxy, ═O, ═S, NO₂, N(CH₃)₂, halogen, amino, or SH.

An “alkynyl” group refers to an unsaturated hydrocarbon group containing at least one carbon-carbon triple bond, including straight-chain, branched chain, and cyclic groups. Preferably, the alkynyl group has 1 to 12 carbons. More preferably it is a lower alkynyl of from 1 to 7 carbons. The alkynyl group may be substituted or unsubstituted. When substituted, the substituted group(s) is preferably hydroxyl, cyano, alkoxy, ═O, ═S, NO₂, N(CH₃)₂, amino, or SH.

“Alkylene” means a linear saturated divalent hydrocarbon radical of one to six carbon atoms or a branched saturated divalent hydrocarbon radical of three to six carbon atoms, e.g., methylene, ethylene, 2,2-dimethylethylene, propylene, 2-methylpropylene, butylene, pentylene, and the like.

An “alkoxy” group refers to an “˜O-alkyl” group, where “alkyl” is defined above. Examples of alkoxy moieties include, but are not limited to, methoxy, ethoxy, isopropoxy, and the like.

“Alkoxyalkyl” means a moiety of the formula R^{a}—O—R^{b}—, where R^{a} is alkyl and R^{b} is alkylene as defined herein. Exemplary alkoxyalkyl groups include, by way of example, 2-methoxyethyl, 3-methoxypropyl, 1-methyl-2-methoxyethyl, 1-(2-methoxyethyl)-3-methoxypropyl, and 1-(2-methoxyethyl)-3-methoxypropyl.

“Aryl” means a monovalent cyclic aromatic hydrocarbon moiety consisting of a mono-, bi- or tricyclic aromatic ring. The aryl group can be optionally substituted as defined herein. Examples of aryl moieties include, but are not limited to, optionally substituted phenyl, naphthyl, phenanthryl, fluorenyl, indenyl, pentalenyl, azulenyl, oxydiphenyl, biphenyl, methylenediphenyl, aminodiphenyl, diphenylsulfidyl, diphenylsulfonyl, diphenylisopropylidenyl, benzodioxanyl, benzofuranyl, benzodioxylyl, benzopyranyl, benzoxazinyl, benzoxazinonyl, benzopiperadinyl, benzopiperazinyl, benzopyrrolidinyl, benzomorpholinyl, methylenedioxyphenyl, ethylenedioxyphenyl, and the like, including partially hydrogenated derivatives thereof.

“Arylalkyl” and “Aralkyl,” which may be used interchangeably, mean a radical-R^{a}R^{b} where R^{a} is an alkylene group and R^{b} is an aryl group as defined herein; e.g., phenylalkyls such as benzyl, phenylethyl, 3-(3-chlorophenyl)-2-methylpentyl, and the like are examples of arylalkyl.

“Heteroalkyl” means an alkyl radical as defined herein wherein one, two or three hydrogen atoms have been replaced with a substituent independently selected from the group consisting of —OR^{a}, —NR^{b}R^{c}, and —S(O)ₙR^{d} (where n is an integer from 0 to 2), with the understanding that the point of attachment of the heteroalkyl radical is through a carbon atom, wherein R^{a} is hydrogen, acyl, alkyl, cycloalkyl, or cycloalkylalkyl; R^{b} and R^{c} are independently of each other hydrogen, acyl, alkyl, cycloalkyl, or cycloalkylalkyl; and when n is 0, R^{d} is hydrogen, alkyl, cycloalkyl, or cycloalkylalkyl, and when n is 1 or 2, R^{d} is alkyl, cycloalkyl, cycloalkylalkyl, amino, acylamino, monoalkylamino, or dialkylamino. Representative examples include, but are not limited to, 2-hydroxyethyl, 3-hydroxypropyl, 2-hydroxy-1-hydroxymethylethyl, 2,3-dihydroxypropyl, 1-hydroxymethylethyl, 3-hydroxybutyl, 2,3-dihydroxybutyl, 2-hydroxy-1-methylpropyl, 2-aminoethyl, 3-aminopropyl, 2-methylsulfonylethyl, amino sulfonylmethyl, aminosulfonylethyl, amino sulfonylpropyl, methylaminosulfonylmethyl, methylaminosulfonylethyl, methylaminosulfonylpropyl, and the like.

As used herein, an “acyl group” means a linear, branched, or cyclic substituent having a carbonyl group which is attached to either an oxygen atom, e.g., of a hydroxyl group, or a nitrogen atom, e.g., of an amino group. An acyl group can include an alkoxy group, an alkyl group, an aryl group, an arylalkyl group, an ester group, an ether group a heterocyclic group, a vinyl group, and combinations thereof. An acyl group also may be substituted with substituents such as alkanoyloxy groups, alkenyl groups, alkylsilyl groups, alkysulfonyl groups, alkylsulfoxy groups, alkylthio groups, alkynyl groups, amino groups such as mono- and di-alkylamino groups and mono- and di-arylamino groups, amide groups, carboxy groups, carboxyalkoxy groups, carboxyamide groups, carboxylate groups, haloalkyl groups, halogens, hydroxyl groups, nitrile groups, nitro groups, phosphate groups, siloxy groups, sulfate groups, sulfonamide groups, sulfonyloxy groups, and combination of these. It should be understood that an acyl group also can be an amino protecting group or a hydroxyl protecting group. As a hydroxyl protecting group, an acyl group may form an ester or carbonate. As an amino protecting group, an acyl group may form an amide or a carbamate. Examples of acyl groups include, but are not limited to, alkoyl groups, aroyl groups, arylalkoyl groups, vinoyl groups. Preferred acyl groups are benzoyl, ethanoyl, tigloyl, or 2-methyl-2-butenoyl, 2-methyl-1-propenoyl, hexanoyl, butyrl, 2-methylbutyryl, phenylacetyl, propanoyl, furoyl, and tert-butyloxycarbonyl.

An “allyl” group is an alkene hydrocarbon group with the formula H₂C═CH—CH₂—. It is made up of a vinyl group, CH₂═CH—, attached to a methylene —CH₂. For example allyl alcohol has the structure H₂C═CH—CH₂OH. Another example of a simple allyl compound is allyl chloride. Compounds containing the allyl group are often referred to as being allylic. Substituted versions of the parent allyl group, such as the trans-but-2-en-1-yl or crotyl group (CH₃CH═CH—CH₂—), may also be referred to as allylic groups.

“Benzyl” means a substituent or molecular fragment possessing the structure C₆H₅CH₂—.

Terminology related to “protecting”, “deprotecting” and “protected” functionalities occurs throughout this application. Such terminology is well understood by persons of skill in the art and is used in the context of processes which involve sequential treatment with a series of reagents. In that context, a protecting group refers to a group which is used to mask a functionality during a process step in which it would otherwise react, but in which reaction is undesirable. The protecting group prevents reaction at that step, but may be subsequently removed to expose the original functionality. The removal or “deprotection” occurs after the completion of the reaction or reactions in which the functionality would interfere. Thus, when a sequence of reagents is specified, as it is in the processes of the invention, the person of ordinary skill can readily envision those groups that would be suitable as “protecting groups”. Suitable groups for that purpose are discussed in standard textbooks in the field of chemistry, such as Protective Groups in Organic Synthesis by T. W. Greene (John Wiley & Sons, New York, 1991), which is incorporated herein by reference. Particular attention is drawn to the chapters entitled “Protection for the Hydroxyl Group, Including 1,2- and 1,3-Diols” (pages 10-86).

The abbreviations Me, Et, Ph, Tf, Ts and Ms represent methyl, ethyl, phenyl, trifluoromethanesulfonyl, toluenesulfonyl and methanesulfonyl, respectively. A comprehensive list of abbreviations utilized by organic chemists (i.e., persons of ordinary skill in the art) appears in the first issue of each volume of the Journal of Organic Chemistry. The list, which is typically presented in a table entitled “Standard List of Abbreviations” is incorporated herein by reference.

While it may be possible for the compounds of the invention to be administered as the raw chemical, it is preferable to present them as a pharmaceutical composition. According to a further aspect, the present invention provides a pharmaceutical composition comprising a compound of the invention or a pharmaceutically acceptable salt or solvate thereof, together with one or more pharmaceutical carriers thereof and optionally one or more other therapeutic ingredients. The carrier(s) must be “acceptable” in the sense of being compatible with the other ingredients of the formulation and not deleterious to the recipient thereof.

As used herein, the term “solvate” refers to a complex of variable stoichiometry formed by a solute (in this invention, a lycorine compound, or a salt or physiologically functional derivative thereof) and a solvent. Such solvents, for the purpose of the invention, should not interfere with the biological activity of the solute. Non-limiting examples of suitable solvents include, but are not limited to water, methanol, ethanol, and acetic acid. Preferably the solvent used is a pharmaceutically acceptable solvent. Non-limiting examples of suitable pharmaceutically acceptable solvents include water, ethanol, and acetic acid. Most preferably the solvent used is water.

As used herein, the term “physiologically functional derivative” refers to any pharmaceutically acceptable derivative of a compound of the present invention that, upon administration to a mammal, is capable of providing (directly or indirectly) a compound of the present invention or an active metabolite thereof. Such derivatives, for example, esters and amides, will be clear to those skilled in the art, without undue experimentation. Reference may be made to the teaching of Burger's Medicinal Chemistry And Drug Discovery, 5^{th} Edition, Vol 1: Principles and Practice, which is incorporated herein by reference to the extent that it teaches physiologically functional derivatives.

As used herein, the term “effective amount” means that amount of a drug or pharmaceutical agent that will elicit the biological or medical response of a tissue, system, animal, or human that is being sought, for instance, by a researcher or clinician. The term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function. For use in therapy, therapeutically effective amounts of a lycorine compound of the present invention, as well as salts, solvates, and physiological functional derivatives thereof, may be administered as the raw chemical. Additionally, the active ingredient may be presented as a pharmaceutical composition.

Pharmaceutical compositions of the present invention comprise an effective amount of one or more lycorine compound or lycorine compound derivative, or additional agent dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains at least one lycorine compound or lycorine compound derivative, or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The lycorine compound or lycorine compound derivative may comprise different types of carriers depending on whether it is to be administered in solid, liquid or aerosol form, and whether it needs to be sterile for such routes of administration as injection. The present invention can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The lycorine compound or lycorine compound derivative may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present invention, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in the composition include buffers, amino acids such as glycine and lysine, carbohydrates such as dextrose, mannose, galactose, fructose, lactose, sucrose, maltose, sorbitol, mannitol, etc.

In further embodiments, the present invention may concern the use of a pharmaceutical lipid vehicle compositions that include the lycorine compound or lycorine compound derivative, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the lycorine compound or lycorine compound derivative, may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present invention administered to a subject (e.g., an animal or human patient) can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, an active compound may comprise between about 2% to about 75% of the weight of the unit, or between about 25% to about 60%, for example, and any range derivable therein. Naturally, the amount of active compound(s) in each therapeutically useful composition may be prepared in such a way that a suitable dosage will be obtained in any given unit dose of the compound. Factors such as solubility, bioavailability, biological half-life, route of administration, product shelf life, as well as other pharmacological considerations will be contemplated by one of ordinary skill in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In preferred embodiments of the present invention, the lycorine compound or lycorine compound derivative are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792,451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001, which is incorporated by reference herein. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present invention may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

In further embodiments, the lycorine compound or lycorine compound derivative may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,753,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

In other preferred embodiments of the invention, the lycorine compound or lycorine compound derivative may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

In addition, compounds according to the present invention may be administered alone or in combination with other agents, including other compounds of the present invention. Certain compounds according to the present invention may be effective for enhancing the biological activity of certain agents according to the present invention by reducing the metabolism, catabolism or inactivation of other compounds and as such, are co-administered for this intended effect.

In a particularly preferred pharmaceutical composition and method for treating flavivirus infection, an inhibitory effective amount of at least one compound according to the present invention in pharmaceutical dosage form is administered to a patient suffering from such an infection to treat the infection and alleviate the symptoms of such infection.

As indicated, compounds according to the present invention may be administered alone or in combination with other agents, especially including other compounds of the present invention or compounds which are otherwise disclosed as being useful for the treatment of Hepatitis B virus (HBV), Hepatitis C virus (HCV), Yellow Fever virus, Dengue Virus, Japanese Encephalitis, West Nile virus and related flavivirus infection, such as those relevant compounds and compositions which are disclosed in the following United States patents, which are incorporated by reference herein: U.S. Pat. Nos. 5,922,757; 5,830,894; 5,821,242; 5,610,054; 5,532,215; 5,491,135; 5,179,084; 4,902,720; 4,898,888; 4,880,784; 5,929,038; 5,922,857; 5,914,400; 5,922,711; 5,922,694; 5,916,589; 5,912,356; 5,912,265; 5,905,070; 5,892,060; 5,892,052; 5,892,025; 5,883,116; 5,883,113; 5,883,098; 5,880,141; 5,880,106; 5,876,984; 5,874,413; 5,869,522; 5,863,921; 5,863,918; 5,863,905; 5,861,403; 5,852,027; 5,849,800; 5,849,696; 5,847,172; 5,627,160; 5,561,120; 5,631,239; 5,830,898; 5,827,727; 5,830,881 and 5,837,871, among others. The compounds disclosed in the above-referenced patents may be used in combination with the present compounds for their additive activity or treatment profile against Hepatitis B virus (HBV), Hepatitis C virus (HCV), Yellow Fever virus, Dengue Virus, Japanese Encephalitis, West Nile virus and related flavivirus infections and, in certain instances, for their synergistic effects in combination with compounds of the present invention. Preferred secondary or additional compounds for use with the present compounds are those which do not inhibit Hepatitis B virus (HBV), Hepatitis C virus (HCV), Yellow Fever virus, Dengue Virus, Japanese Encephalitis, West Nile virus and related flavivirus infection by the same mechanism as those of the present invention.

The compounds according to the present invention may be produced by synthetic methods which are readily known to those of ordinary skill in the art and include various chemical synthetic methods which are presented in detail hereinbelow.

During chemical synthesis of the various compositions according to the present invention, one of ordinary skill in the art will be able to practice the present invention without engaging in undue experimentation. In particular, one of ordinary skill in the art will recognize the various steps that should be performed to introduce a particular substituent at the desired position of the napthalene group or benzene ring, following the specific teachings of the present invention and well known principles of aromatic chemistry. These steps are well known in the art. In addition, chemical steps which are taken to “protect” functional groups such as hydroxyl or amino groups, among others, as well as “de-protect” these same functional groups, will be recognized as appropriate within the circumstances of the syntheses. As set forth herein, a large number of protecting groups may be used in the present invention. In the case of the introduction of any one or more acyl groups onto a hydroxyl group, standard techniques, well known by those of ordinary skill, may be used. Synthesis of other prodrug forms of the compounds of the present invention may also be synthesized by well known methods in the art.

### EXAMPLES

The following examples are intended to illustrate particular embodiments of the present invention, but are by no means intended to limit the scope of the present invention.

### Example 1

### A Single-Amino Acid Substitution in West Nile Virus 2K Peptide Between NS4A and NS4B Confers Resistance to Lycorine, a Flavivirus Inhibitor

Lycorine potently inhibits flaviviruses in cell culture. At 1.2-1 μM concentration, lycorine reduced viral titers of West Nile (WNV), dengue, and yellow fever viruses by 10²- to 10⁴-fold. However, the compound did not inhibit an alphavirus (Western equine encephalitis virus) or a rhabdovirus (vesicular stomatitis virus), indicating a selective antiviral spectrum. The compound exerts its antiviral activity mainly through suppression of viral RNA replication. A Val→Met substitution at the 9th amino acid position of the viral 2K peptide (spanning the endoplasmic reticulum membrane between NS4A and NS4B proteins) confers WNV resistance to lycorine, through enhancement of viral RNA replication. Initial chemistry synthesis demonstrated that modifications of the two hydroxyl groups of lycorine can increase the compound's potency, while reducing its cytotoxicity. Taken together, the results have established lycorine as a flavivirus inhibitor for antiviral development. The lycorine-resistance results demonstrate a direct role of the 2K peptide in flavivirus RNA synthesis.

### Example 2

### Materials and Methods

The following Materials and Methods relate to the experimental study summarized in Example 1 (above).

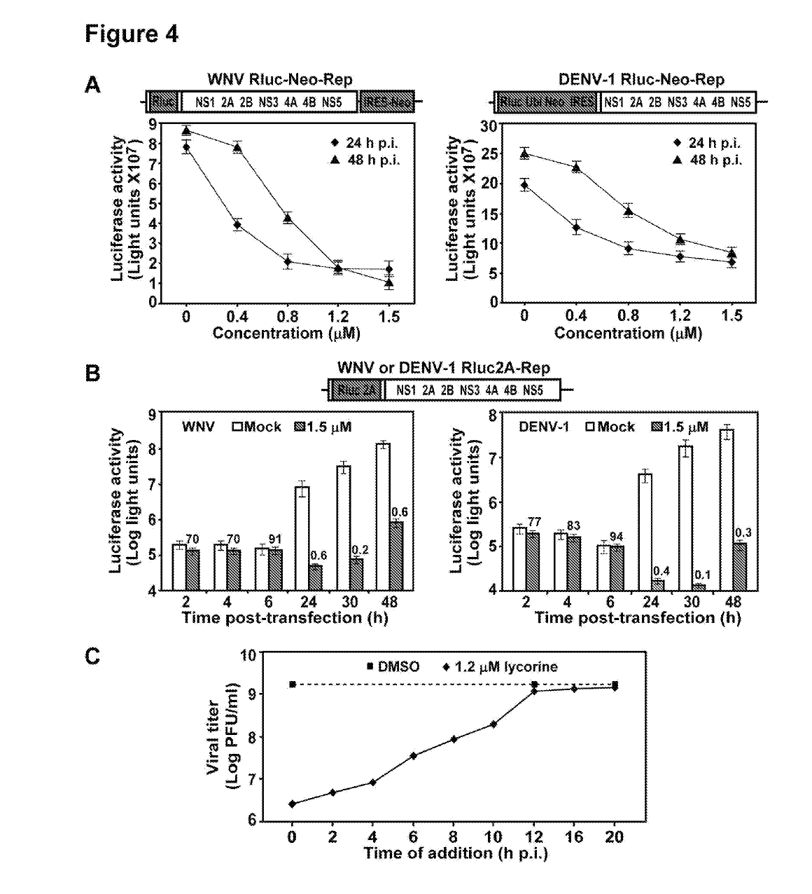

Cells and Viruses: Baby hamster kidney cells (BHK-21) and African green monkey kidney cells (Vero) were cultured in Dulbecco modified Eagle medium (DMEM) with 10% fetal bovine serum in 5% CO₂ at 37° C. Aedes albopictus C6/36 cells were grown in Eagle's minimal essential medium (EMEM) with 10% FBS and 1% non-essential amino acid at 28° C. A reporting Vero cell line containing a persistently replicating WNV or DENV-1 replicon (Rluc-Neo-Rep; FIG. 4A) was cultured in DMEM with 10% FBS and 1 mg/ml of G418. WNV was derived from a full-length infectious cDNA clone of an epidemic strain 3356 (Shi et al., 2002). YFV (17D vaccine strain), DENV-2 (New Guinea C strain), WEEV (strain Cova 746), and VSV (New Jersey serotype) were used for antiviral assays, as described previously (Puig-Basagoiti et al., 2006).

Lycorine and Analogues: Lycorine was purchased from BIOMOL Research Laboratories Inc. A panel of seven lycorine analogues (Table 1) was synthesized based on previous methodology: compounds 1180 (Lee et al., 2007), 1181 (Nakagawa, Uyeo, and Yajima, 1956), 1193 (Lee et al., 2007), 1194 (Lee et al., 2007), 1197 (Shu et al., 2002), and 1200 (Nakagawa, Uyeo, and Yajima, 1956). Illustrative methods for synthesizing compounds 1198 and 1200 are found herein at Examples 5 and 6, respectively. The structure and purity (>95%) of each compound were established by nuclear magnetic resonance. All compounds were dissolved in dimethyl sulfoxide (DMSO), and were tested at 1% DMSO final concentration. Mock-treated reactions were incubated with 1% DMSO without lycorine or any of its analogues.

TABLE 1
Antiviral Activities of Lycorine Analogues

| Com- | | CC₅₀ | EC₅₀ |
|---|---|---|---|
| pound | Structure | (μM)^{a} | (μM)^{b} |
| Lycorine | 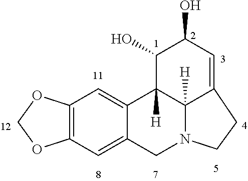 | 24 | 0.23 |
| 1180 | 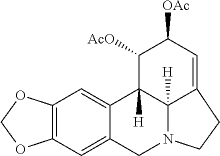 | 110 | 1.49 |
| 1181 | 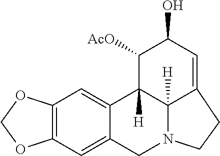 | 66 | 0.86 |
| 1193 | 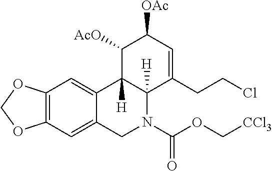 | >300 | >300 |
| 1194 | 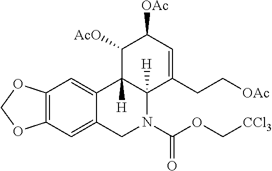 | >300 | 72 |
| 1197 | 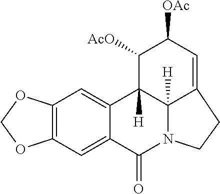 | >300 | 300 |
| 1198 | 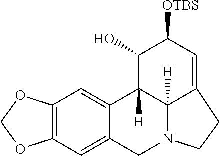 | 78 | 0.73 |
| 1200 | 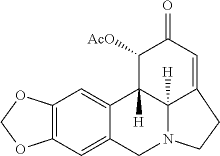 | >300 | 0.19 |

^{a}CC₅₀ values were derived from Vero cells using an MTT assay.
^{b}EC₅₀ values were derived from viral titer reduction assays. Vero cells were infected with WNV (0.1 MOI) in the presence of various concentrations of each compound. Viral titers at 42 h p.i. were determined by plaque assays, as described in the Materials and Methods (Example 2).

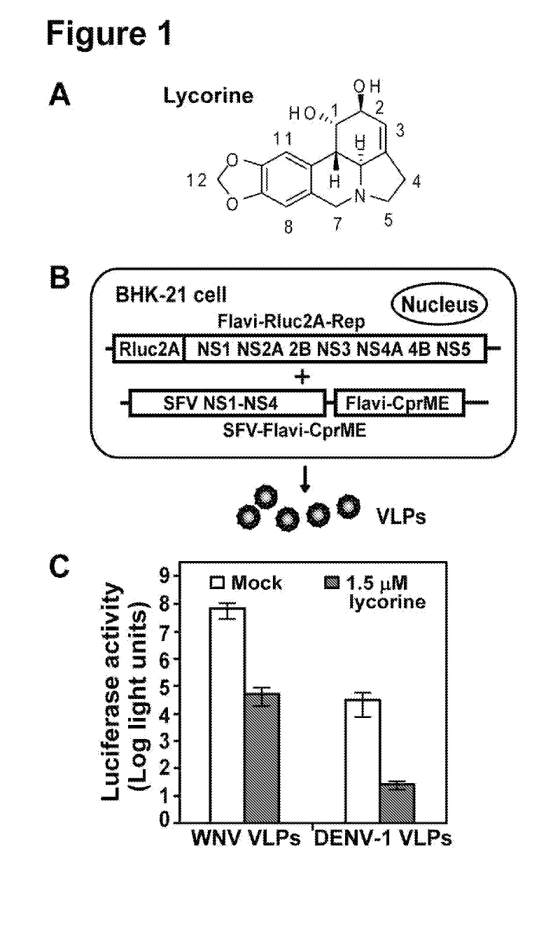

VLP Infection Assay: VLPs of WNV and DENV-1 were prepared by trans-supply of viral structural proteins to replicon RNAs. Each replicon contains a Renilla luciferase (Rluc) and foot-and-mouth disease virus 2A sequence in the position where the viral structural genes (CprME) were deleted (Rluc2A-Rep; FIG. 1B). The construction and characterization of the WNV and DENV-1 Rluc2A-Rep were reported previously (Lo et al., 2003; Puig-Basagoiti et al., 2006). The structural genes of each virus were cloned into an SFV expression vector (Invitrogen) (Liljestrom and Garoff, 1991) at a unique Bam HI site, resulting in SFV-CprME (FIG. 1B). A Kozak sequence and a stop codon were engineered at the 5′ and 3′ ends of the CprME fragment, respectively. A double-transfection protocol was used to generate the VLPs (Khromykh, Varnayski, and Westaway, 1998; Puig-Basagoiti et al., 2005). Briefly, BHK-21 cells (8×10⁶) were first electroporated with 10 μg of Rluc2A-Rep RNA in a 0.4-cm cuvette with the GenePulser apparatus (Bio-Rad) using settings of 0.85 kV and 25 μF, pulsing three times at 3-sec intervals. The transfected cells were resuspended in DMEM with 10% FBS and incubated at 37° C. with 5% CO₂ for 24 h. The replicon-transfected cells were then electroporated with 10 μg of SFV-CprME RNA of the corresponding virus, at the settings identical to those used for the first transfection. At 24 h after the second transfection, culture supernatants were collected and centrifuged to remove cellular debris. The VLPs in the supernatants were aliquoted and stored at −80° C. The titers of VLPs (in focus-forming units [FFU]/ml) were estimated by infection of Vero cells in a four-chamber Lab-Tek Chamber Slide (Nalge Nunc International) with serial dilutions of the culture fluid, followed by counting of immunofluorescence assay (IFA)-positive cell foci at 18 and 24 h p.i. for WNV and DENV-1, respectively. For IFA, immune mouse ascites fluid of WNV or DENV-1 (American Type Culture Collection) and goat anti-mouse immunoglobulin G conjugated with Texas Red were used as primary and secondary antibodies, respectively.

For VLP-based antiviral assays, a monolayer of naive Vero cells (2×10⁴ cells per well in 96-well plate) was infected at 1 FFU/cell for WNV VLP, and at 0.01 FFU/cell for DENV-1 VLP. Lycorine was immediately added to the VLP-infected cells. At 24 h p.i., the cells were washed twice with cold PBS, lysed in 20 μA of 1×Renilla luciferase lysis buffer for 20 min, and assayed for luciferase activities using a Renilla luciferase assay kit (Promega). The luciferase signals were measured with a Veritas Microplate Luminometer (Promega). Average results of three or more independent experiments are presented here.

Reporting Replicon Cell Line Assay: Vero cells containing persistently replicating replicons of WNV (Lo, Tilgner, and Shi, 2003) or DENV-1 (Puig-Basagoiti et al., 2006) were constructed previously. Each replicon contained two reporter genes: a Renilla luciferase (Rluc) and a neomycin phosphotransferase gene (Neo), resulting in Rluc-Neo-Rep (FIG. 4A). For antiviral assays, Rluc-Neo-Rep-containing Vero cells (2×10⁴ in 100 μl) were seeded per well of 96-well plates in DMEM with 10% FBS without G418. Lycorine was added to the medium at 24 h post-seeding. After 24 or 48 h of lycorine-treatment, the cells were lysed and assayed for luciferase activities as described above.

Transient Replicon Assay: A transient replicon assay was used to quantify compound-mediated inhibition of viral translation and suppression of RNA synthesis (Deas et al., 2005). Briefly, 10 μg of Rluc2A-Rep RNA of WNV or DENV-1 (FIG. 4B) was electroporated into BHK-21 cells (8×10⁶) as described above. The transfected cells were suspended in 25 ml of DMEM with 10% FBS. Cell suspension (1 ml) was seeded into 12-well plates, immediately treated with lycorine, and assayed for luciferase activities at 2, 4, and 6 h p.t. (representing viral translation), and at 24, 30, and 48 h p.t. (representing RNA synthesis). For quantification of compound-mediated inhibition, relative luciferase activities were presented, with the luciferase activity derived from the mock-treated cells set as 100%.

Viral Titer Reduction Assay and Cytotoxicity Assay: Viral titer reduction assays were performed to examine the antiviral activities of lycorine and its analogues in WNV, DENV-2, YFV, WEEV and VSV. Approximately 6×10⁵Vero cells per well were seeded in a 12-well plate. After incubation for 24 h, the cells were infected with individual virus (0.1 MOI) and treated immediately with one of the compounds at the indicated concentration. For WNV, DENV-2, YFV, and WEEV, samples of culture medium were collected at 42 h post-infection. For VSV, culture medium was collected at 16 h post-infection. All collected samples were stored at −80° C., and viral titers were determined by plaque assays on Vero cells. A double-overlayer protocol was followed for plaque assays (Puig-Basagoiti et al., 2006). All assays were performed in triplicate. Cytotoxicity of each compound was examined using a cell proliferation-based MTT assay (American Type Culture Collection) as described previously (Puig-Basagoiti et al., 2006).

Generation and Sequencing of WNV Resistant to Lycorine: Three independent lineages of lycorine-resistant WNV were generated by passaging of the WT WNV (derived from an infectious cDNA clone) on Vero cells, with increasing concentrations of lycorine. For the first six passages, Vero cells in 12-well plates were infected with WNV (derived each time from the previous passage) at an MOI of 0.1 in the presence of 0.8 μM lycorine or 1% DMSO (as a negative control). Passages 7 to 12 were selected with 1.2 μM lycorine. For each passage, viral supernatants were harvested at 42 h p.i.; viral titers were quantified by plaque assays; viruses were analyzed in resistance assays to monitor the improvement of resistance, through comparison of the viral titers from the lycorine-treated infections with the viral titers from the mock-treated infections. The selections were terminated at passage 12, when no further improvement of the resistance was observed (see details in Results, as set forth in Example 3).

Lycorine-resistant WNVs from passage 12 were subjected to genome-length sequencing for identification of accumulated mutation(s). Virion RNAs were extracted from culture supernatants using RNeasy kits (QIAGEN). Viral RNAs were amplified by RT-PCR using SuperScript III one-step RT-PCR kits (Invitrogen). The PCR products were gel-purified and subjected to DNA sequencing. Sequences of the 5′- and 3′-terminal nucleotides of the viral genomes were determined by rapid amplification of cDNA ends (RACE). The 5′RACE was performed using the FirstChoice RLM-RACE kit (Ambion). The 3′RACE was performed as previously described (Tilgner and Shi, 2004).

Construction of cDNA Plasmids of WNV Containing Various Mutations: WNV genome-length cDNA clones with specific mutations were constructed by using a modified pFLWNV (Shi et al., 2002) and two shuttle vectors. Shuttle vector A was constructed by engineering the Bam HI-Sph I fragment from the pFLWNV (representing the upstream end of the T7 promoter (for RNA transcription of genome-length RNA) to nucleotide position 3627 of the WNV genome; GenBank No. AF404756) into the pACYC177 vector containing a modified cloning cassette (Zhou et al., 2007). A QuikChange II XL site-directed mutagenesis Kit (Stratagene) was used to engineer the mutations in the E gene into the shuttle vector A. The mutated DNA fragment was cut-and-pasted back into the pFLWNV clone at the Bam HI and Stu I sites (nucleotide position 2591). Shuttle vector B was constructed by engineering Kpn I-Xba I fragment (representing nucleotide 5341 through the 3′ end of the genome) into a pcDNA3.1(+) vector. The G6871A mutation was engineered into the shuttle vector B using the QuikChange II XL site-directed mutagenesis Kit. The mutated DNA fragment was pasted back into the pFLWNV clone and into the cDNA clone of WNV Rluc2A replicon at the Bsi WI and Spe I sites (nucleotide positions 5780 to 8022). All constructs were verified by DNA sequencing.

RNA Transcription and Transfection: Both genome-length RNA and replicon RNA were in vitro transcribed from corresponding cDNA plasmids that were linearized with Xba I. A T7 mMessage mMachine kit (Ambion) was used for RNA synthesis described before (Shi, Tilgner, and Lo, 2002). Both replicon and genome-length RNAs were electroporated into BHK-21 cells as described above. For transfection of genome-length RNA, culture fluids were collected every 24 h until apparent cytopathic effect was observed (day 4 to day 5 post-transfection). The viruses in the supernatants were aliquoted and stored at −80° C.

### Example 3

### Results

The following Results relate to the experimental study summarized in Example 1 (above).

Identification of Lycorine as an Inhibitor of WNV and DENV-1: Lycorine (FIG. 1A) has been reported to have antiviral activities, as noted herein above). To test whether lycorine inhibits flaviviruses, the compound was initially screened using a viral-like particle (VLP)-based infection assay. As depicted in FIG. 1B, VLPs of WNV and DENV-1 were prepared by trans-supply viral structural proteins (CprME; through an alphavirus Semliki Forest virus [SFV] expression vector) to package corresponding replicon RNAs containing a luciferase reporter (Rluc2A-Rep). The titers of the VLPs were estimated to be 2.5×10⁶ and 2.4×10³ FFU/ml for WNV and DENV-1, respectively. Vero cells were infected with 1 FFU/cell of WNV VLP or with 0.01 FFU/cell of DENV-1 VLP (due to the low titer of DENV-1 VLP). The infected cells were treated with 1.5 μM lycorine or were mock-treated with 1% DMSO. At 24 h post-infection (p.i.), lycorine reduced the luciferase signals by 1,400- and 1,200-fold in the WNV and DENV-1 VLP-infected cells, respectively (FIG. 1C). Higher concentrations of lycorine were also tested. The results indicate that lycorine inhibits WNV and DENV-1.

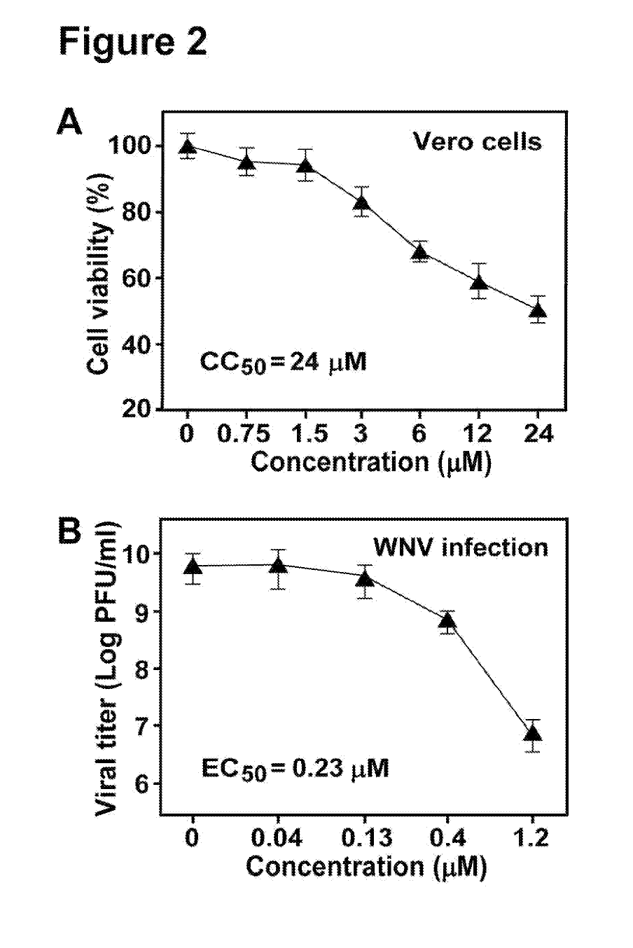

Inhibition of an Epidemic Strain of WNV at Noncytotoxic Concentrations: In order to exclude the possibility that the above-described antiviral activity was due to compound-mediated cytotoxicity (FIG. 2A), an MTT assay was performed to determine the cytotoxicity of lycorine. Vero cells were incubated with various concentrations of lycorine for 48 h; cell viability was indicated by cellular metabolism of MTT tetrazolium salt. The CC₅₀ (50% cytotoxic concentration) value of the compound was estimated to be 24 μM. At 1.5 μM, cell viability was >90%. Therefore, all following experiments were performed at ≦1.5 μM of lycorine.

Next, the antiviral activity of lycorine was assayed using an authentic WNV infection assay. Vero cells were infected with an epidemic strain of WNV (0.1 MOI). The infections were treated with various concentrations of the compound, and assayed for viral yields in culture medium at 42 h p.i. (FIG. 2B). The compound suppressed viral titer in a dose-responsive manner. At 1.2 μM, the compound reduced the viral titer by 910-fold. The EC₅₀ (50% effective concentration) value was estimated to be 0.23 μM. The results demonstrate that lycorine inhibits WNV at noncytotoxic concentrations.

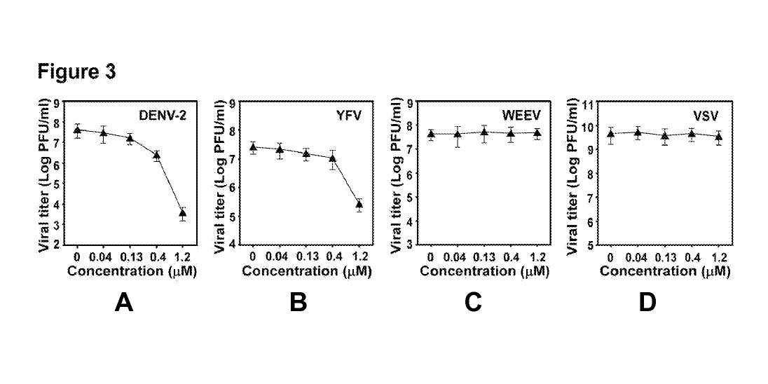

Selective Antiviral Spectrum: To examine the antiviral spectrum of lycorine, viral titer reduction assays were performed using other flaviviruses (DENV-2 and YFV), a plus-strand RNA alphavirus (Western equine encephalitis virus, WEEV), and a negative-strand RNA rhabdovirus (vesicular stomatitis virus, VSV). Lycorine inhibited both DENV-2 and YFV (FIG. 3). At 1.2 μM, the compound reduced viral titers of DENV-2 and YFV by 1.1×10⁴- and 98-fold, respectively. In contrast, the compound suppressed titers of neither WEEV nor VSV at the tested concentrations. The latter results further suggest that lycorine at 1.2 μM is not cytotoxic. Overall, the results indicate that the antiviral spectrum of the compound is selective for flaviviruses.

Inhibitory Steps of Viral Infection: Given that lycorine inhibits VLP-mediated infection (FIG. 1C), the compound could block steps of viral entry and/or replication. To pinpoint the step(s) of compound inhibition, the compound was tested in the Vero cell lines containing persistently replicating replicon of WNV or DENV (Rluc-Neo-Rep; FIG. 4A). Incubation of the Rluc-Neo-Rep cells with 1.5-1 μM lycorine for 24 h or 48 h led to reduced luciferase activities, suggesting that the compound inhibits viral translation and/or RNA synthesis.

A transient replicon system (Rluc2A-Rep; FIG. 4B) was then used to differentiate between inhibition of viral translation and inhibition of RNA synthesis. Previous studies showed that transfection of BHK-21 cells with the Rluc2A-Rep of WNV or DENV-1 produces two luciferase peaks, one at 1 to 8 h post-transfection (p.t.), and another at >20 h p.t.; these respectively represent viral translation and RNA replication (Lo et al., 2003; Puig-Basagoiti et al., 2006). As shown in FIG. 4B, whereas lycorine suppressed early luciferase signals (at 2, 4, and 6 h p.t.) by ≦30%, it reduced late luciferase signals (at 24, 30 and 48 h p.t.) by >99%. Taken together, the results suggest that lycorine weakly inhibits viral translation, but strongly suppresses RNA synthesis.

Time-of-Addition Analysis: A time-of-addition experiment was performed to further elucidate the mode-of-action of lycorine (FIG. 4C). Vero cells were synchronously infected with WNV (Puig-Basagoiti et al., 2006). Lycorine (1.2 μM) was added to the infected cells at various time points post-infection. Viral titers in the culture medium were determined at 20 h post-infection. As controls, 1% DMSO was added to infected cells at 0, 12, or 20 h p.i., for estimation of its effect on viral yield. The results showed that the inhibitory effect of lycorine on viral titer gradually diminished when the compound was added at time points up to 10 h p.i.; the compound completely lost its antiviral activity when added after 10 h post-infection. These results agree with the transient replicon results, which indicated that lycorine weakly inhibits viral translation, but strongly suppresses RNA synthesis.

Lycorine Does Not Inhibit WNV Protease, NTPase, MTase, or RdRp Activities: To identify potential antiviral target(s), lycorine was directly tested in previously established enzyme assays, using recombinant proteins of WNV, including protease (with NS2B), NTPase, MTase, and RdRp (Ray et al., 2006; Wong et al., 2003). None of the enzyme activities were suppressed by the compound at concentrations up to 100 μM. The results suggest that the compound does not directly target the enzyme functions of the viral NS3 or NS5 proteins.

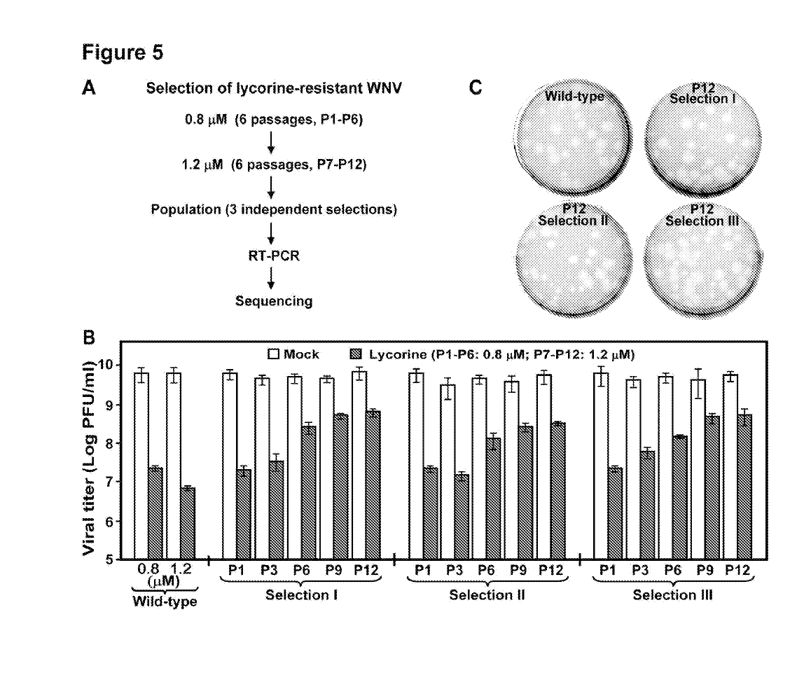

Selection and Characterization of Lycorine-Resistant WNV: As an alternative means by which to identify antiviral target(s), lycorine-resistant WNV were selected by culturing wild-type (WT) virus in the presence of increasing concentrations of the compound (FIG. 5A). Three independent selections (I-III) were performed. For each selection, a total of 12 passages were carried out, with the first 6 passages (P1-P6) selected at 0.8 μM lycorine and the last 6 passages (P7-P12) selected at 1.2 μM lycorine. Viruses from each passage were assayed for their resistance, through comparison of viral titers from mock-treated infection with viral titers from lycorine-treated infection (harvested at 42 h p.i.). FIG. 5B shows representative data from such resistance assays for P1, P3, P6, P9, and P12. Viral resistance gradually improved from P1 to P10; no further improvement was observed from P10 to P12 (FIG. 5B). The selections were therefore terminated at P12. Notably, the P12 viruses did not show complete resistance to lycorine. At 1.2 μM, the compound reduced viral titers of P12 virus-infected cells by approximately 10-fold. In contrast, the compound suppressed viral titers of the WT virus-infected cells by about 1,000-fold at the same concentration (FIG. 5B). The results indicated that the resistance of the P12 viruses increased by approximately 100-fold over that of the WT. Similar results were obtained for all three independent selections.

Plaque morphologies of the WT and lycorine-resistant P12 viruses were compared in agar containing no lycorine (FIG. 5C). Plaques derived from the P12 viruses were slightly more heterogeneous in size than the plaques derived from the WT virus, indicating that the P12 viruses were composed of quasispecies. However, the majority of the plaques from the P12 viruses are similar in size to the plaques derived from the WT virus. Collectively, the results demonstrated that WNVs partially resistant to lycorine can be reproducibly selected in cell culture.

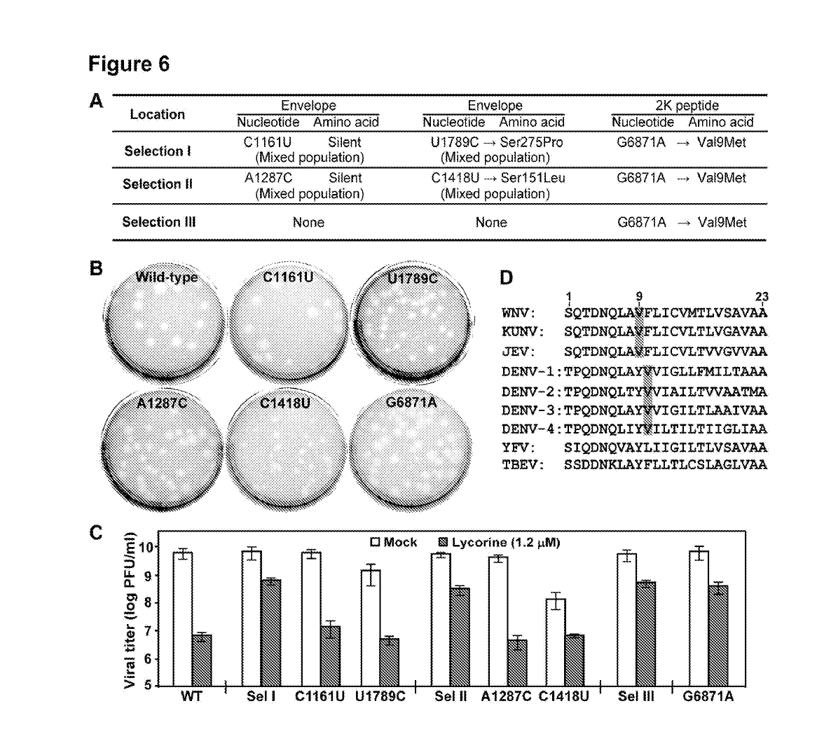

Sequencing of Lycorine-Resistant WNV: The complete genomes of P12 viruses from the three independent selections were sequenced, to identify potential resistance mutation(s). Population sequencing was performed by directly sequencing the RT-PCR products amplified from the RNA extracted from the P12 culture supernatants (FIG. 5A). All selections had accumulated a consensus nucleotide change from G to A at position 6871 (G6871A), resulting in an amino acid substitution from Val to Met at position 9 (Val9Met) in the 2K peptide. No other mutation was detected from Selection III. For Selections I and II, two additional, but distinct, nucleotide mutations were recovered in the E gene: one mutation was silent, while the other one led to an amino-acid change (FIG. 6A). Sequencing chromatograms indicated that the E mutations existed in mixed populations, as represented by both WT and mutant nucleotide peaks at the specific positions. As a negative control, viruses passaged in 1% DMSO did not give rise to any mutations.

Identification of a Single-Amino Acid Mutation in the 2K Peptide as a Resistance Determinant Two panels of recombinant viruses were prepared to identify the lycorine-resistance determinant. The first panel of viruses contained the mutations in the E gene. Each of the four mutations in the E region was individually engineered into an infectious cDNA clone of WNV. Transfection of BHK-21 cells with the genome-length RNAs resulted in four mutant viruses (FIG. 6B): C1161U and U1789C (derived from Selection I), and A1287C and C1418U (derived from Selection II). The four mutant viruses exhibited different plaque morphologies: the two viruses containing the silent mutations (C1161U and A1287C) yielded plaques similar to those of the WT virus, whereas the mutant viruses containing amino acid changes in the E protein (C1418U and U1789C) generated smaller plaques than those of the WT virus. Resistance assays showed that, after treatment of infected cells (0.1 MOI) with lycorine (1.2 μM lycorine for 42 h), none of the four mutants yielded viral titers that were significantly higher than the titers of the WT virus (FIG. 6C). The results indicate that the E mutations are not responsible for resistance. Notably, the smaller plaques (FIG. 6B) and the lower titers from mock-treated infections for mutant viruses U1789C and C1418U (FIG. 6C) suggest that the amino acid changes in the E gene negatively affect viral replication.

The second panel of viruses was prepared to examine the mutation G6817A in the 2K peptide. The plaque morphology of G6871A virus was similar to that of the WT virus (FIG. 6B). Remarkably, the G6871A virus showed a resistance level close to those of the P12 viruses from all three selections (FIG. 6C). Sequencing of the G6871A mutant virus indicated that the engineered mutation was retained without extra changes; furthermore, the G6871A mutation was retained after passaging the mutant virus in Vero cells for five rounds (total 10 days). These results demonstrate that the G6871A mutation in the 2K peptide is the major determinant for lycorine resistance. Sequence alignment (FIG. 6D) indicates that the mutated Val at amino-acid position 9 of the 2K peptide (total of 23 amino acids) is conserved among the members of the JEV-serocomplex; and a Val at position 10 is conserved among the members of the DENV-serocomplex; whereas no Val is found at a similar position in the 2K peptides from members of the YFV- and TBEV-serocomplexes.

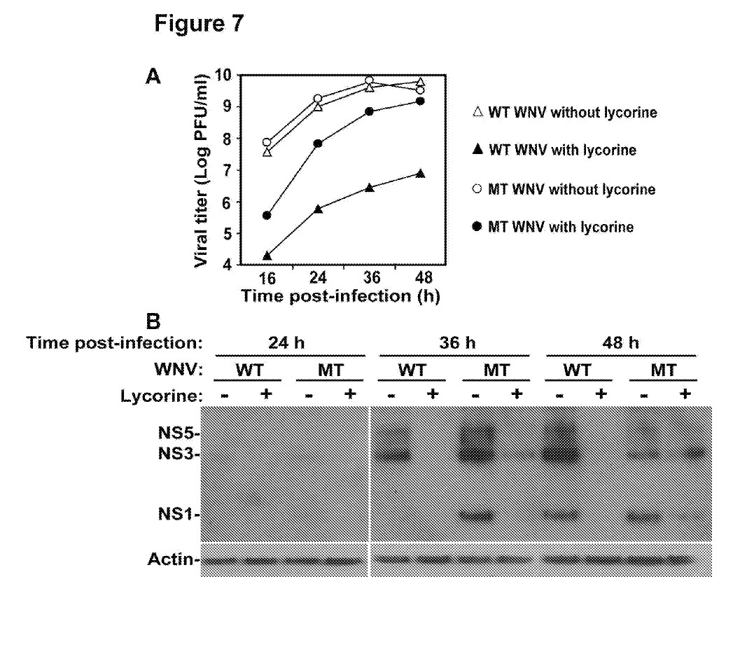

Replication Kinetics of WT and 2K-Mutant Viruses in the Presence and Absence of Lycorine: The G6871A mutation was further characterized by comparing the replication kinetics between the WT and mutant (MT) viruses in the presence and absence of lycorine inhibitor. Vero cells were synchronizely infected with the WT and the G6871A MT viruses, and treated with or without 1.2 μM lycorine. As expected, the compound inhibited WT virus more dramatically than it did on the MT virus (FIG. 7A). Western blotting analysis showed that, at 16 h p.i., no viral proteins could be detected in cells infected with either WT or mutant viruses. The expression levels of viral NS1, NS3, and NS5 increased from 24 to 36 h p.i., but decreased at 48 h p.i. The decrease in protein expression at 48 h p.i. was due to cytopathic effect (i.e., cell lysis). At each time point, lycorine treatment suppressed viral protein expression. These results confirm the critical role of the G6871A mutation in lycorine resistance.

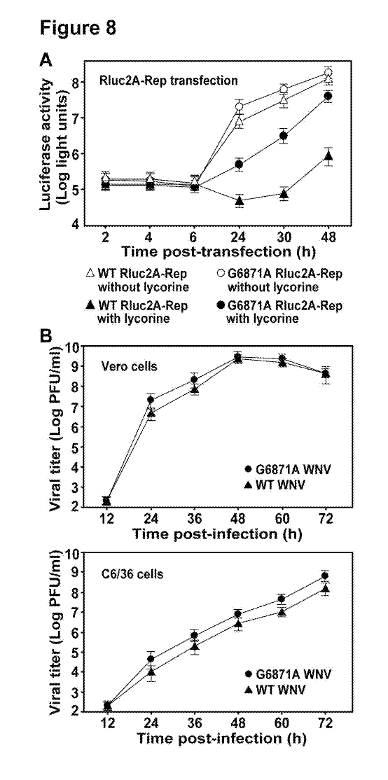

Enhancement of RNA Replication by the 2K Peptide Mutation: To further validate the contribution of the G6871A change to resistance, the mutation was engineered into a reporting replicon (Rluc2A-Rep) of WNV. In the absence of lycorine, the mutant and WT replicons yielded equal levels of luciferase activities at 2, 4, and 6 h p.t.; in contrast, the mutant replicon exhibited luciferase signals 2.5-, 2-, and 1.4-fold higher than the WT replicon at 24, 30, and 48 h p.t., respectively (FIG. 8A). These results demonstrate that the 2K peptide mutation does not affect viral translation, but it instead enhances RNA synthesis, especially at the early replication stage. In the presence of inhibitor (1.5 μM), the luciferase signals from the WT and mutant replicons were reduced. The reporting signals at 2, 4, and 6 h p.t. were slightly suppressed (≦30%) for both replicons; however, the luciferase activities at 24, 30, and 48 h p.t. from the WT replicon were more dramatically suppressed by the compound than were the activities from the mutant replicon. These results again demonstrate that the single-amino acid change in the 2K peptide is responsible for lycorine resistance.

To further examine the effect of the G6871A mutation in the 2K peptide on viral replication, the growth kinetics of the WT and mutant viruses were compared. Vero and mosquito C6/36 cells were each infected with WT or mutant virus (0.05 MOI) and were then monitored for viral yields. In Vero cells, the mutant virus produced titers 4.2- and 2.7-fold higher than those of the WT virus at 24 and 36 h p.i., respectively; the difference in viral titer was reduced to <1.4-fold after 48 h p.i. (FIG. 8B). In C3/36 cells, the mutant virus generated titers 2.8- to 4.4-fold higher than those of the WT virus at all tested time points. For both Vero and C6/36 cells, similar titers of the WT and mutant viruses were detected at 12 h p.i.; these low viral titers were most likely derived from the input viruses during inoculation, and are not substantially composed of progeny viruses. These results, together with the transient replicon data (FIG. 8A), strongly indicate that the 2K peptide mutation enhances viral replication. It should be noted that the replication enhancement was not evident when the plaque sizes were compared between the WT and mutant viruses (FIG. 6B).

Improvement of Antiviral Profile of Lycorine Compounds: Chemistry studies were initiated by synthesizing seven lycorine analogues (Table 1). The analogues were each examined for cytotoxicity and antiviral activity. Three regions of the parental lycorine compound were modified. (i) One or two hydroxyl groups at the C1 and C2 positions of lycorine were substituted with other chemical groups. All seven analogues that contained these modifications showed CC₅₀ values higher than the value for parental lycorine. Among them, compound 1200, in which the hydroxyl group at the C₁-position is acetylated and the C2 hydroxyl group is oxidized to form a carbonyl group, slightly increased the potency (EC₅₀=0.19 μM), while significantly reducing cytotoxicity (CC₅₀>300 μM, the highest tested concentration). As expected, the G6871A mutant virus was resistant to compound 1200. (ii) The pyrrolidine ring of lycorine was opened (compounds 1193 and 1194). (iii) C7 of lycorine was oxidized to a carbonyl group (compound 1197). Compounds from (ii) and (iii) modifications had lower antiviral potencies. The results clearly indicate that modifications at the two hydroxyl groups of lycorine could be used to improve the antiviral profile.

### Example 4

### Discussion

The following Discussion section relates to the experimental study summarized in Example 1 (above).

A number of approaches have been reported for development of flavivirus antiviral agents (Shi, 2008). The goals of this study were to establish the anti-flavivirus activity of lycorine and to analyze lycorine-resistant flavivirus in cell culture. Previous studies showed that lycorine inhibits poliovirus (Ieven et al., 1982), SARS-CoV (Li et al., 2005), herpes simplex virus (Renard-Nozaki et al., 1989), and vaccinia virus (Zhou et al., 2003). The current study has extended the antiviral spectrum to flaviviruses. In addition, it was shown that lycorine does not inhibit an alphavirus, WEEV, and a rhabdovirus, VSV (FIG. 3). The latter results, together with findings of an early study showing that lycorine does not inhibit HIV-1 at a noncytotoxic concentration (Szlavik et al., 2004), indicate the selectivity of the antiviral spectrum of the compound. As a proof-of-principle, it was shown that modifications of the lycorine compound, especially at the two hydroxyl groups, could dramatically reduce its cytotoxicity, while improving its potency (Table 1). The results suggest that lycorine represents one class of inhibitor that could be further developed for flavivirus therapeutics.

Three WNV luciferase-reporting replicon-based assays were used to dissect the inhibitory step(s) of lycorine. First, a VLP-infection-based assay allowed identification of inhibitors of viral entry and replication (FIG. 1C). Second, use of Replicon-containing cell lines allowed screening for inhibitors of viral replication (FIG. 4A). Third, a transient replicon assay allowed differentiation between inhibitors of translation and inhibitors of RNA synthesis (FIG. 4B). Analysis of the compound in the above systems indicated that whereas lycorine only weakly reduces viral translation (≦30%, as indicated by luciferase activity), it significantly suppresses RNA synthesis (>99%, as indicated by luciferase activity). The reduction of RNA synthesis could be caused by the compound-mediated suppression of viral translation. Alternatively, lycorine could suppress both translation and RNA synthesis, leading to the dramatic reduction of RNA synthesis. To differentiate between the above two possibilities, a time-of addition experiment was performed. The results showed that lycorine gradually declined its anti-WNV activity, when the time of addition was varied from 0 to 10 h p.i.; the compound completely lost the inhibitory activity when added at >10 p.i. (FIG. 4C). A single round of flavivirus infection is about 12 h in duration, with viral translation peaking around 2-6 h p.i. and RNA synthesis peaking around 7-12 h p.i. (Chambers et al., 1990; Puig-Basagoiti et al., 2005). If lycorine only inhibited the step of translation, it would have completely lost its antiviral activity when added at a time point earlier than 10 h p.i. (i.e., at 6 h p.i.). Therefore, the time-of-addition results clearly indicate that the compound inhibits a step beyond viral translation. These data, together with the identification of the resistance determinant in the 2K peptide (FIG. 6), led to the conclusion that lycorine suppresses WNV mainly through suppression of viral RNA synthesis.

The data presented herein are in contrast to poliovirus results, which indicated that ˜90% of viral translation was suppressed by 10 μg/ml (equivalent to 31 μM) lycorine (Ieven et al., 1982). Since the cytotoxicity of lycorine at 31 μM was not shown in the poliovirus study, experiments are needed to exclude the possibility that the strong inhibition of viral translation was due to compound-mediated cytotoxicity. The mode-of-action analysis was performed at ≦1.5 μM of lycorine, a concentration that allowed >90% cell viability. In this system, lycorine at 31 μM decreased the viability of Vero cells by ˜50% (FIG. 2A).

Two approaches were taken to identify potential the viral target(s) of lycorine. The first approach was to test the compound in biochemical enzyme assays using recombinant protease (with NS2B), NTPase, RdRp, and MTase proteins. None of the enzyme activities were suppressed by lycorine. The second approach was to select compound-resistant WNV. Resistant WNVs in cell culture were selected. Engineering of the mutations (recovered from the resistant viruses) into an infectious clone (FIG. 6) and a replicon (FIG. 8A) of WNV allowed for the mapping of the resistance determinant to a single amino-acid change (Val9Met) in the 2K peptide. It should be noted that the resistance results do not necessarily indicate that the 2K peptide is the direct target for lycorine. The 2K peptide mutation could exert its resistance phenotype through enhancement of viral replication so that, in the presence of inhibitor, viral replication could still be sustained at a level sufficient to generate virus. The latter scenario is supported by the observations that the 2K peptide mutation enhanced RNA synthesis of WNV replicon as well as the growth kinetics of WNV in Vero and C6/36 cells (FIG. 8). However, it should be pointed out that the 2K mutation-mediated enhancement of viral replication only partially contributes to the lycorine resistance. The latter conclusion was supported by the results that the 2K mutation increased replication by only 2 to 4-fold in the absence of lycorine (FIG. 8B), whereas the same mutation increased replication by >100-fold in the presence of the inhibitor (FIG. 7A).

The question of how the 2K peptide mutation enhances WNV RNA replication was considered. Flavivirus 2K peptide spans the ER membrane with its N- and C-terminal tails on the cytoplasmic and ER lumen sides, respectively (Miller et al., 2007). The cleavage at the 2K-NS4B junction by host signalase requires a prior cleavage at the NS4A-2K junction by viral NS2B/NS3 protease (Lin et al., 1993). The regulated cleavages at the NS4A-2K-NS4B sites play a role in rearranging cytoplasmic membranes (Miller et al., 2007; Roosendaal et al., 2006). The exact function of the 2K peptide in induction of membrane arrangement may differ among flaviviruses. In KUNV, individual expression of NS4A-2K resulted in membrane rearrangements that most resembling virus-induced structures, while removal of the 2K domain led to a less profound membrane rearrangement (Roosendaal et al., 2006). In contrast, expression of DENV-2 NS4A without 2K peptide resulted in altered membranes resembling virus-induced structures, whereas expression of NS4A-2K did not induce comparable membrane arrangement (Miller et al., 2007). Since the rearranged membranes form the scaffold for the viral replication complex, the Val9Met mutation may affect the 2K peptide-mediated membrane alterations, leading to an increase in RNA replication.

Alternatively, since the cleavages of NS4A-2K-4B intermediate are critical for the NS4A- and NS4B-mediated inhibition of interferon signaling (Munoz-Jordan et al., 2005), the Val9Met mutation in the 2K peptide may affect the processing of the NS4A-2K-4B polyprotein, resulting in a more efficient defense against the host immune response. The weakened interferon response allowed the virus to replicate to a higher level. This hypothesis is supported by the observations that the same 2K peptide mutation was recovered from the WNV that was able to perform superinfections (Zhang and Shi, unpublished data). In the later study, it was found that the 2K peptide mutant virus showed significantly improved capability to replicate in cells that contain persistently replicating replicon of WNV. One caveat to this hypothesis is that the lycorine resistance study was performed in Vero cells, in which the interferon gene has been deleted (Mosca and Pitha, 1986). More experiments are required to dissect the molecular mechanism of the 2K peptide that is operative in flavivirus replication or in evasion of the host immune response.

### Example 5

### Compound 1198

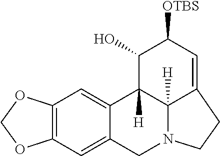

Lycorine (1.05 g, 3.65 mmol) was dissolved in hot dry DMF (100 mL). To this solution at rt were added imidazole (350 mg, 5.14 mmol), 4-dimethylaminopyridine (DMAP, 12 mg, 0.098 mmol) and tert-butyldimethylsilyl chloride (TBSCl, 665 mg, 4.41 mmol). The solution was allowed to stir overnight at rt and then concentrated. The residue was purified by silica gel column chromatography (2-6% MeOH/CHCl₃ plus 0.1% ammonia) to give compound 1198 as a yellow-brown solid (1.10 g, 75%). ¹H NMR (CD₃OD, 600 MHz) δ 6.82 (s, 1H), 6.64 (s, 1H), 5.92 (d, J=1.2 Hz, 1H), 5.91 (d, J=1.2 Hz, 1H), 5.46 (s, 1H), 4.40 (s, 1H), 4.28 (t, J=1.5 Hz, 1H), 4.10 (d, J=13.8 Hz, 1H), 3.52 (dd, J=13.8, 1.2 Hz, 1H), 3.31-3.28 (m, 1H), 2.87 (d, J=10.8 Hz, 1H), 2.78 (d, J=10.2 Hz, 1H), 2.70-2.63 (m, 1H), 2.62-2.54 (m, 1H), 2.41 (dd, J=18.0, 9.0 Hz, 1H), 0.93 (s, 9H), 0.20 (s, 3H), 0.16 (s, 3H). ESIMS calcd for C₂₂H₃₂NO₄Si 402.21 (M+H)⁺. Found 402.25.

### Example 6

### Compound 1200

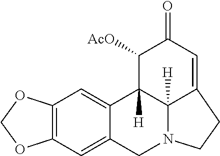

To the solution of 1-O-Acethyllycorine (0.3 g, 0.9 mmol) in CHCl₃ (30 mL) was added activated MnO₂ (2 g). Mixture was stirred at rt for 16 h, filtered, and filter cake was washed thoroughly with CHCl₃ and evaporated to leave crude mixture. TLC on silica gel in CHCl₃:MeOH 9:1 revealed product+substrate ˜1:1. After purification by preparative TLC in CHCl₃:MeOH 9:1 product 60 mg was crystallized from EtOH:hexane. Yield: 100 mg (34%) of crystalline compound. ¹H NMR (600 MHz, cdcl₃) δ 6.70 (s, 1H), 6.55 (s, 1H), 5.97 (d, J=2.8 Hz, 2H), 5.90 (dd, J=6.7, 1.3 Hz, 2H), 4.15 (d, J=14.0 Hz, 1H), 3.58 (d, J=14.0 Hz, 1H), 3.43 (dt, J=9.0, 4.5 Hz, 1H), 3.24 (d, J=10.0 Hz, 1H), 3.15 (d, J=10.0 Hz, 1H), 2.88-2.78 (m, 2H), 2.50 (q, J=8.7 Hz, 1H), 1.93 (s, 3H).

### REFERENCES CITED

Citation of a reference herein shall not be construed as an admission that such reference is prior art to the present invention. The following references, to the extent that they provide exemplary procedural or other details supplementary to those set forth herein, are specifically incorporated herein by reference:
Ackermann, M., and Padmanabhan, R. (2001). De novo synthesis of RNA by the dengue virus RNA-dependent RNA polymerase exhibits temperature dependence at the initiation but not elongation phase. J. Biol. Chem. 276(43), 39926-37.
Best, S. M., Morris, K. L., Shannon, J. G., Robertson, S. J., Mitzel, D. N., Park, G. S., Boer, E., Wolfinbarger, J. B., and Bloom, M. E. (2005) Inhibition of interferon-stimulated JAK-STAT signaling by a tick-borne flavivirus and identification of NS5 as an interferon antagonist. J. Virol. 79(20), 12828-39.
Chambers, T. J., Hahn, C. S., Galler, R., and Rice, C. M. (1990). Flavivirus genome organization, expression, and replication. Annu. Rev. Microbiol. 44, 649-88.
Chattopadhyay, U., Chaudhuri, L., Das, S., Kumar, Y., and Ghosal, S. (1984). Activation of lymphocytes by lycorine-1-O-beta-D-glucoside. Pharmazie 39(12), 855-6.
De Leo, P., Dalessandro, G., De Santis, A., and Arrigoni, O. (1973). Metabolic responses to lycorine in plants. Plant Cell Physiol. 14, 487-496.
Deas, T. S., Binduga-Gajewska, I., Tilgner, M., Ren, P., Stein, D. A., Moulton, H. M., Iversen, P. L., Kauffman, E. B., Kramer, L. D., and Shi, P.-Y. (2005). Inhibition of flavivirus infections by antisense oligomers specifically suppressing viral translation and RNA replication. J. Virol. 4599-4609.
Egloff, M. P., Benarroch, D., Selisko, B., Romette, J. L., and Canard, B. (2002). An RNA cap (nucleoside-2′-O-)-methyltransferase in the flavivirus RNA polymerase NS5: crystal structure and functional characterization. EMBO J. 21(11), 2757-68.
Falgout, B., Miller, R. H., and Lai, C. J. (1993). Deletion analysis of dengue virus type 4 nonstructural protein NS2B: identification of a domain required for NS2B-NS3 protease activity. J. Virol. 67(4), 2034-42.
Gubler, D., Kuno, G., and Markoff, L. (2007). Flaviviruses, p. 1153-1253. In D. M. Knipe and P. M. Howley (ed), Fields virology, 5th., vol. 1. Lippincott William & Wilkins, Philadelphia, Pa.
Guo, J., Hayashi, J., and Seeger, C. (2005). West nile virus inhibits the signal transduction pathway of alpha interferon. J. Virol. 79(3), 1343-50.
Guyatt, K. J., Westaway, E. G., and Khromykh, A. A. (2001). Expression and purification of enzymatically active recombinant RNA-dependent RNA polymerase (N55) of the flavivirus Kunjin. J. Virol. Methods 92(1), 37-44.
Ieven, M., Vlietinck, A. J., Vanden Berghe, D. A., Totte, J., Dommisse, R., Esmans, E., and Alderweireldt, F. (1982). Plant antiviral agents. III. Isolation of alkaloids from Clivia miniata Regel (Amaryllidaceae). J Nat Prod 45(5), 564-73.
Jones, C., Patkar, C., and Kuhn, R. (2005). Construction and applications of yellow fever virus replicons. Virology 331(2), 247-59.
Khromykh, A. A., Varnayski, A. N., and Westaway, E. G. (1998). Encapsidation of the flavivirus Kunjin replicon RNA by using a complementation system providing Kunjin virus structural proteins in trans. J. Virol. 72, 5967-5977.
Kramer, L., Li, J., and Shi, P. (2007). West Nile virus. The Lancet Neurology 6, 171-182.
Kummerer, B. M., and Rice, C. M. (2002). Mutations in the yellow fever virus nonstructural protein NS2A selectively block production of infectious particles. J. Virol. 76(10), 4773-4784.
Lee, S.-S., Venkatesham, U., Rao, C. P., Lam, S.-H., and Lin, J.-H. (2007). Preparation of secolycorines against acetylcholinesterase. Bioorganic & Med. Chem. 15, 1034-1043.
Leung, D., Schroder, K., White, H., Fang, N.-X., Stoermer, M., Abbenante, G., Martin, J., PR, Y., and Fairlie, D. (2001). Activity of recombinant dengue 2 virus NS3 protease in the presence of a truncated NS2B co-factor, small peptide substrates, and inhibitors. J. Biol. Chem. 276, 45762-45771.
Li, H., Clum, S., You, S., Ebner, K. E., and Padmanabhan, R. (1999). The serine protease and RNA-stimulated nucleoside triphosphatase and RNA helicase functional domains of dengue virus type 2 NS3 converge within a region of 20 amino acids. J. Virol. 73(4), 3108-16.
Li, S. Y., Chen, C., Zhang, H. Q., Guo, H. Y., Wang, H., Wang, L., Zhang, X., Hua, S, N., Yu, J., Xiao, P. G., Li, R. S., and Tan, X. (2005). Identification of natural compounds with antiviral activities against SARS-associated coronavirus. Antiviral Res 67(1), 18-23.
Liljestrom, P., and Garoff, H. (1991). A new generation of animal cell expression vectors based on the Semliki Forest virus replicon. Bio/Technology 9(12), 1356-61.
Lin, C., Amberg, S. M., Chambers, T. J., and Rice, C. M. (1993). Cleavage at a novel site in the NS4A region by the yellow fever virus NS2B-3 proteinase is a prerequisite for processing at the downstream 4A/4B signalase site. J. Virol. 67(4), 2327-35.
Lindenbach, B. D., Thiel, H.-J., and Rice, C. M. (2007). “Flaviviridae: The Virus and Their Replication.” Fourth ed. Fields Virology (D. M. Knipe, and P. M. Howley, Eds.) Lippincott William & Wilkins.
Liu, J., Hu, W. X., He, L. F., Ye, M., and Li, Y. (2004). Effects of lycorine on HL-60 cells via arresting cell cycle and inducing apoptosis. FEBS Lett 578(3), 245-50.
Liu, J., Li, Y., Tang, L. J., Zhang, G. P., and Hu, W. X. (2007). Treatment of lycorine on SCID mice model with human APL cells. Biomed Pharmacother 61(4), 229-34.
Liu, W., Wang, X., Mokhonov, V., Shi, P., Randall, R., and Khromykh, A. (2005) Inhibition of interferon signaling by the New York 99 strain and Kunjin subtype of West Nile virus involves blockage of STAT1 and STAT2 activation by nonstructural proteins. J. Virol. 79(3), 1934-42.
Liu, W. J., Chen, H. B., and Khromykh, A. A. (2003). Molecular and functional analyses of Kunjin virus infectious cDNA clones demonstrate the essential roles for NS2A in virus assembly and for a nonconservative residue in NS3 in RNA replication. J. Virol. 77(14), 7804-13.
Lo, L., Tilgner, M., Bernard, K., and Shi, P.-Y. (2003). Functional analysis of mosquito-borne flavivirus conserved sequence elements within 3′ untranslated region of West Nile virus using a reporting replicon that differentiates between viral translation and RNA replication. J. Virol. 77(18), 10004-10014.
Lo, L., Tilgner, M., and Shi, P.-Y. (2003). A potential high-throughput assay for screening inhibitors of West Nile virus replication. J. Virol. 77(23), 12901-12906.
Miller, S., Kastner, S., Krijnse-Locker, J., Buhler, S., and Bartenschlager, R. (2007). The non-structural protein 4A of dengue virus is an integral membrane protein inducing membrane alterations in a 2K-regulated manner. J Biol Chem 282(12), 8873-82.
Mosca, J. D., and Pitha, P. M. (1986). Transcriptional and posttranscriptional regulation of exogenous human beta interferon gene in simian cells defective in interferon synthesis. Mol. Cell. Biol. 6(6), 2279-83.
Munoz-Jordan, J. L., Laurent-Rolle, M., Ashour, J., Martinez-Sobrido, L., Ashok, M., Lipkin, W. I., and Garcia-Sastre, A. (2005) Inhibition of Alpha/Beta Interferon Signaling by the NS4B Protein of Flaviviruses. J. Virol. 79(13), 8004-13.
Munoz-Jordan, J. L., Sanchez-Burgos, G. G., Laurent-Rolle, M., and Garcia-Sastre, A. (2003). Inhibition of interferon signaling by dengue virus. Proc. Natl. Acad. Sci. USA 100(24), 14333-8.
Nakagawa, Y., Uyeo, S., and Yajima, H. (1956). The double bond in lycorine. Chemistry & Industry (London, United Kingdom), 1238-9.
Puig-Basagoiti, F., Deas, T. S., Ren, P., Tilgner, M., Ferguson, D. M., and Shi, P.-Y. (2005). High-throughput assays using luciferase-expressing replicon, virus-like particle, and full-length virus for West Nile virus drug discovery. Antimicrob. Agent. Chemother. 49(12), 4980-4988.
Puig-Basagoiti, F., Tilgner, M., Forshey, B., Philpott, S., Espina, N., Wentworth, Goebel, S., Masters, P. S., Falgout, B., Ren, P., Ferguson, and Shi, P. Y. (2006). Triaryl pyrazoline compound inhibits flavivirus RNA replication. Antimicrob. Agents Chemother. 50(4), 1320-9.

Ray, D., Shah, A., Tilgner, M., Guo, Y., Zhao, Y., Dong, H., Deas, T., Zhou, Y., Li, H., and Shi, P. (2006). West nile virus 5′-cap structure is formed by sequential guanine N-7 and ribose 2′-O methylations by nonstructural protein 5. J. Virol. 80(17), 8362-70.
Renard-Nozaki, J., Kim, T., Imakura, Y., Kihara, M., and Kobayashi, S. (1989). Effect of alkaloids isolated from Amaryllidaceae on herpes simplex virus. Res Virol 140(2), 115-28.
Roosendaal, J., Westaway, E. G., Khromykh, A., and Mackenzie, J. M. (2006). Regulated cleavages at the West Nile virus NS4A-2K-NS4B junctions play a major role in rearranging cytoplasmic membranes and Golgi trafficking of the NS4A protein. J Virol 80(9), 4623-32.
Shi, P.-Y. (2008). Novel therapeutics against West Nile virus. West Nile Encephalitis Virus Infection: Viral pathogenesis and the Host Immune Response In M. S. Diamond (ed), Springer Publisher (In press).
Shi, P. Y., Tilgner, M., and Lo, M. K. (2002). Construction and characterization of subgenomic replicons of New York strain of West Nile virus. Virology 296, 219-233.
Shi, P. Y., Tilgner, M., Lo, M. K., Kent, K. A., and Bernard, K. A. (2002). Infectious cDNA clone of the epidemic West Nile virus from New York City. J. Virol. 76(12), 5847-56.
Shu, P. Y., Chen, L. K., Chang, S. F., Yueh, Y. Y., Chow, L., Chien, L. J., Chin, C., Yang, H. H., Lin, T. H., and Huang, J. H. (2002). Potential application of nonstructural protein NS1 serotype-specific immunoglobulin G enzyme-linked immunosorbent assay in the seroepidemiologic study of dengue virus infection: correlation of results with those of the plaque reduction neutralization test. Journal of Clinical Microbiology 40(5), 1840-4.
Szlavik, L., Gyuris, A., Minarovits, J., Forgo, P., Molnar, J., and Hohmann, J. (2004). Alkaloids from Leucojum vernum and antiretroviral activity of Amaryllidaceae alkaloids. Planta Med 70(9), 871-3.
Tan, B. H., Fu, J., Sugrue, R. J., Yap, E. H., Chan, Y. C., and Tan, Y. H. (1996). Recombinant dengue type 1 virus NS5 protein expressed in Escherichia coli exhibits RNA-dependent RNA polymerase activity. Virology 216(2), 317-25.
Tilgner, M., and Shi, P. Y. (2004). Structure and function of the 3′ terminal six nucleotides of the West Nile virus genome in viral replication. J. Virol. 78(15), 8159-71.

Warrener, P., Tamura, J. K., and Collett, M. S. (1993). RNA-stimulated NTPase activity associated with yellow fever virus NS3 protein expressed in bacteria. J. Virol. 67(2), 989-96.
Wengler, G., and Wengler, G. (1991). The carboxy-terminal part of the NS 3 protein of the West Nile flavivirus can be isolated as a soluble protein after proteolytic cleavage and represents an RNA-stimulated NTPase. Virology 184(2), 707-15.
Wong, S. J., Boyle, R. H., Demarest, V. L., Woodmansee, A. N., Kramer, L. D., Li, H., Drebot, M., Koski, R. A., Fikrig, E., Martin, D. A., and Shi, P.-Y. (2003). An immunoassay targeting nonstructural protein 5 to differentiate West Nile virus infection from dengue and St. Louis encephalitis virus infections, and form flavivirus vaccination. J. Clin. Microbiol. 41(9), 4217-23.
Zhou, M., Deng, L., Kashanchi, F., Brady, J. N., Shatkin, A. J., and Kumar, A. (2003). The Tat/TAR-dependent phosphorylation of RNA polymerase II C-terminal domain stimulates cotranscriptional capping of HIV-1 mRNA. Proc. Nat. Acad. Sci. USA 100(22), 12666-71.
Zhou, Y., Ray, D., Zhao, Y., Dong, H., Ren, S., Li, Z., Guo, Y., Bernard, K., Shi, P., and Li, H. (2007). Structure and function of flavivirus NS5 methyltransferase. J. Virol. 81(8), 3891-3903.

What is claimed is:

1. A method of treating a subject infected by a flavivirus, said method comprising:
administering to said subject infected by a flavivirus an effective amount of a compound of the structure (I), (II), or (III), or a pharmaceutically acceptable salt of said compound, optionally in combination with a pharmaceutically acceptable excipient, carrier, or additive, wherein structures (I), (II), and (III) are as follows: 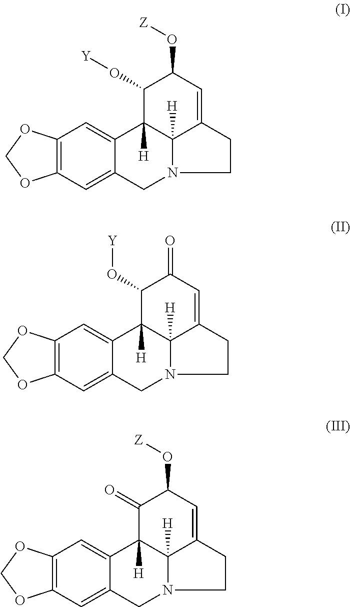
wherein:
Y and Z are each independently selected from the group consisting of H, alkyl, aralkyl, alkoxyalkyl, heteroalkyl, alkenyl, acyl, alkylsilyl, and arylalkylsilyl; or
Y and Z together are alkylidenyl or aralkylidenyl.

2. The method according to claim 1, wherein Y and Z are each independently selected from the group consisting of: H, methoxymethyl (MOM), tetrahydropyranyl (THP), allyl (All), benzyl (Bzyl), t-butyl (tBu), t-butyldimethylsilyl (TBS), t-butyldiphenylsilyl (TBDPS), acetyl (Ac), pivalyl (Piv), and benzoyl (Bzoyl); or
wherein Y and Z together are isopropylidenyl [(CH₃)₂CH] or benzylidenyl [Φ-CH].

3. The method according to claim 2, wherein at least one of Y or Z is selected from the group consisting of: methoxymethyl (MOM), tetrahydropyranyl (THP), allyl (All), benzyl (Bzyl), t-butyl (tBu), t-butyldimethylsilyl (TBS), t-butyldiphenylsilyl (TBDPS), acetyl (Ac), pivalyl (Piv), and benzoyl (Bzoyl).

4. The method according to claim 2, wherein the compound has structure (I), and wherein Y and Z are each Ac.

5. The method according to claim 2, wherein the compound has structure (I), and wherein Y is Ac and Z is H.

6. The method according to claim 2, wherein the compound has structure (I), and wherein Y is H and Z is TBS.

7. The method according to claim 2, wherein the compound has structure (II), and wherein Y is selected from the group consisting of: H, alkyl, aralkyl, alkoxyalkyl, heteroalkyl, alkenyl, acyl, alkylsilyl, and arylalkylsilyl.

8. The method according to claim 2, wherein the compound has structure (II), and wherein Y is selected from the group consisting of: methyl, ethyl, methoxymethyl (MOM), tetrahydropyranyl (THP), allyl (All), benzyl (Bzyl), t-butyl (tBu), t-butyldimethylsilyl (TBS), t-butyldiphenylsilyl (TBDPS), acetyl (Ac), pivalyl (Piv), and benzoyl (Bzoyl).

9. The method according to claim 2, wherein the compound has structure (II), and wherein Y is selected from the group consisting of substituted acetyl and substituted benzoyl.

10. The method according to claim 2, wherein the compound has structure (II), and wherein Y is Ac.

11. The method according to claim 1, wherein said flavivirus is selected from the group consisting of West Nile virus (WNV), dengue virus (DENV), Japanese encephalitis virus (JEV), yellow fever virus (YFV), tick-borne encephalitis virus (TBEV), St. Louis encephalitis virus (SLEV), Alfuy virus (AV), Koutango virus (KV), Kunjin virus (KUNV), Cacipacore virus (CV), Yaounde virus (YV), and Murray Valley encephalitis virus (MVEV).

12. The method according to claim 1, wherein the compound is administered parenterally, orally, subcutaneously, intravenously, intramuscularly, or intradermally.

13. The method according to claim 1, wherein the compound is in the form of a dosage unit.

14. The method according to claim 13, wherein the dosage unit is a tablet, a capsule, or a liquid.

15. A method of preventing a flavivirus infection in a subject, said method comprising:
administering to said subject an effective amount of a compound of the structure (I), (II), or (III), or a pharmaceutically acceptable salt of said compound, optionally in combination with a pharmaceutically acceptable excipient, carrier, or additive, wherein structures (I), (II), and (III) are as follows: 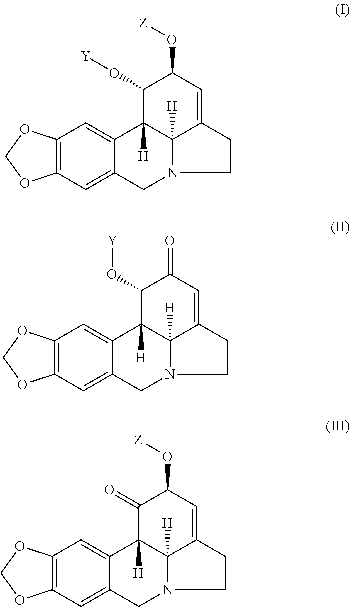
wherein:
Y and Z are each independently selected from the group consisting of H, alkyl, aralkyl, alkoxyalkyl, heteroalkyl, alkenyl, acyl, alkylsilyl, and arylalkylsilyl; or
Y and Z together are alkylidenyl or aralkylidenyl.

16. A method of suppressing viral RNA synthesis of a flavivirus, said method comprising:
providing a compound of the structure (I), (II), or (III) as follows: 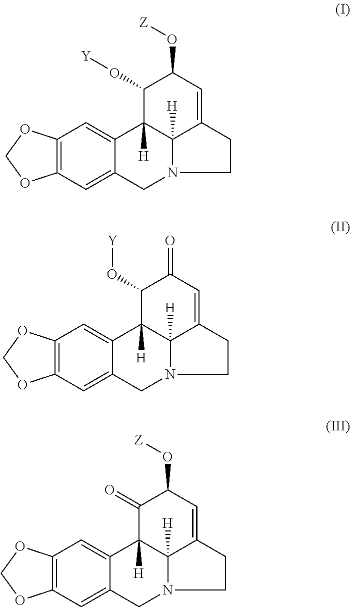
wherein:
Y and Z are each independently selected from the group consisting of H, alkyl, aralkyl, alkoxyalkyl, heteroalkyl, alkenyl, acyl, alkylsilyl, and arylalkylsilyl; or
Y and Z together are alkylidenyl or aralkylidenyl; and
contacting said flavivirus with an effective amount of said compound to suppress the viral RNA synthesis of said flavivirus.

17. The method according to claim 16, wherein said flavivirus is selected from the group consisting of West Nile virus (WNV), dengue virus (DENV), Japanese encephalitis virus (JEV), yellow fever virus (YFV), tick-borne encephalitis virus (TBEV), St. Louis encephalitis virus (SLEV), Alfuy virus (AV), Koutango virus (KV), Kunjin virus (KUNV), Cacipacore virus (CV), Yaounde virus (YV), and Murray Valley encephalitis virus (MVEV).

18. A method for preparing an anti-flavivirus compound for use in the treatment or prophylaxis of a flavivirus infection in a subject, said method comprising:
providing a lycorine compound having a structure of: 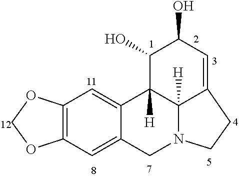
or a precursor of said compound; and
substituting the hydroxyl group at position 1 and/or at position 2 with a protecting group in order to yield an anti-flavivirus agent having a therapeutic index (TI) of 10 or greater, wherein said therapeutic index comprises the ratio of CC₅₀ (uM)/EC₅₀ (uM).

19. The method according to claim 18, wherein said protecting group is selected from the group consisting of: methoxymethyl (MOM), tetrahydropyranyl (THP), allyl (All), benzyl (Bzyl), t-butyl (tBu), t-butyldimethylsilyl (TBS), t-butyldiphenylsilyl (TBDPS), acetyl (Ac), pivalyl (Piv), benzoyl (Bzoyl), substituted acetyl, and substituted benzoyl.

20. An anti-flavivirus compound having a structure of:

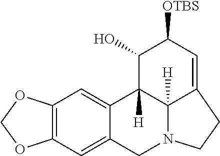

21. The anti-flavivirus compound according to claim 20, wherein the —OH group is protected by a substituent group selected from the group consisting of: methoxymethyl (MOM), tetrahydropyranyl (THP), allyl (All), benzyl (Bzyl), t-butyl (tBu), t-butyldimethylsilyl (TBS), t-butyldiphenylsilyl (TBDPS), acetyl (Ac), pivalyl (Piv), benzoyl (Bzoyl), substituted acetyl, and substituted benzoyl.


SEQUENCE LISTING (9 sequences)

SEQ ID NO: 1 - protein, 23 aa, West Nile Virus
SQTDNQLAVFLICVMTLVSAVAA

SEQ ID NO: 2 - protein, 23 aa, Kunjin Virus
SQTDNQLAVFLICVLTLVGAVAA

SEQ ID NO: 3 - protein, 23 aa, Japanese Encephalitis Virus
SQTDNQLAVFLICVLTVVGVVAA

SEQ ID NO: 4 - protein, 23 aa, Dengue Virus Serotype 1
TPQDNQLAYVVIGLLFMILTAAA

SEQ ID NO: 5 - protein, 23 aa, Dengue Virus Serotype 2
TPQDNQLTYVVIAILTVVAATMA

SEQ ID NO: 6 - protein, 23 aa, Dengue Virus Serotype 3
TPQDNQLAYVVIGILTLAAIVAA

SEQ ID NO: 7 - protein, 23 aa, Dengue Virus Serotype 4
TPQDNQLIYVILTILTIIGLIAA

SEQ ID NO: 8 - protein, 23 aa, Yellow Fever Virus
SIQDNQVAYLIIGILTLVSAVAA

SEQ ID NO: 9 - protein, 23 aa, Tick-Borne Encephalitis Virus
SSDDNKLAYFLLTLCSLAGLVAA